(19) United States
(12) Patent Application Publication
(10) Pub. No.: US 2024/0082830 A1
(43) Pub. Date: Mar. 14, 2024

(54) TRIARYL BORANE CATALYSTS AND METHOD FOR SELECTIVE HYDROSILYLATION OF ESTERS AND LACTONES USING SAID CATALYSTS

(71) Applicant: ALDEXCHEM KFT., Érd (HU)

(72) Inventors: Tibor SOÓS, Budapest (HU); Ádám GYÖMÖRE, Budapest (HU); Ádám DUDÁS, Budapest (HU); Dániel FEGYVERNEKI, Budapest (HU); Máté GYÖNGYÖSI, Debrecen (HU); Petra SOREGI, Budapest (HU); Natália KOLOZSVÁRI, Budapest (HU)

(21) Appl. No.: 18/268,030

(22) PCT Filed: Dec. 16, 2021
(86) PCT No.: PCT/HU2021/050073

§ 371 Date: Jun. 16, 2023

(30) Foreign Application Priority Data
Dec. 18, 2020 (HU) P2000437

(51) Int. Cl. B01J 31/14; C07C 45/41; C07D 307/20; C07F 5/02; C07F 7/18

(52) U.S. Cl. CPC B01J 31/146; C07C 45/41; C07D 307/20; C07F 5/027; C07F 7/1804; B01J 2231/643; B01J 2531/002

(57) ABSTRACT

The present invention relates to a catalytic process for the partial reduction of esters or lactones to silyl acetals, which upon hydrolysis give aldehydes, using silanes as reducing agents, preferably triethylsilane (TESH) or 1,1,3,3-tetramethyldisiloxane (TMDS), in the presence of novel triaryl borane type catalysts. More specifically, the present invention relates to novel triaryl borane type catalyst compounds of formula (I) which can be applied for the partial reduction of an ester or lactone to a silyl acetal. In the formula R₁, R′₁, R₅, R′₅ and R₆ are groups having small steric demand and R10 is a group having large steric demand. The invention also relates to N a method for the preparation of aldehydes or lactols wherein said method comprises the following steps: i) an ester or lactone is reacted with a silane in the presence of a compound of formula (I) to obtain a silyl acetal; ii) the obtained silyl acetal is hydrolysed with acidic or fluoride containing reagent to form an aldehyde or lactol; iii) optionally, the resulting aldehyde or lactol is separated and purified.

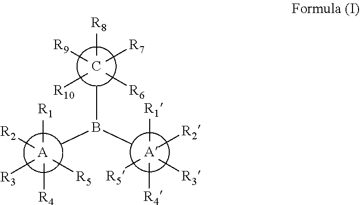

### FIELD OF THE INVENTION

The present invention relates to a catalytic process for the partial reduction of esters or lactones to silyl acetals, which upon hydrolysis give aldehydes or lactols, using silanes as reducing agents, e.g. triethylsilane (TESH), in the presence of novel triaryl borane type catalysts.

More specifically, the present invention relates to novel triaryl borane type catalyst compounds of formula (I) (see below) which can be applied for the partial reduction of an ester or lactone to a silyl acetal. The invention also relates to a method for the preparation of aldehydes or lactols wherein said method comprises the following steps: i) an ester or lactone is reacted with a silane in the presence of a compound of formula (I) to obtain a silyl acetal; ii) the obtained silyl acetal is hydrolysed with acidic or fluoride containing reagent to form an aldehyde or lactol; iii) optionally, the resulting aldehyde or lactol is separated and purified.

### BACKGROUND OF THE INVENTION

Aldehydes and lactols are useful products as such in perfumery industry/agrochemistry, but also important intermediates for the preparation of fine chemicals, especially in the pharmaceutical industry. As esters and lactones are easily available and relatively cheap starting materials, the selective reduction of an ester functional group to the corresponding aldehyde is one of the fundamental reactions in organic chemistry and is used in many chemical processes. To avoid the overreduction to alcohols, the reactions should halt at the acetal intermediates, i.e. the reaction of the silane should happen only with the CO═O function. Until now, hydride reducing agents were exclusively used, such as diisobutyl aluminium hydride (DIBAL-H) or lithium tri-tent-butoxyaluminium hydride. The use of these reagents is costly, as they are required to conduct the reactions at low temperature to minimize overreduction to alcohols. Additionally, they show the disadvantage of high flammability, of violent reaction with water liberating extremely flammable gases, of spontaneous flammability in air and of challenging work up procedure. Nevertheless, when the overreduction to alcohol cannot be avoided with these two reagents, an indirect, two-step protocol is used to obtain the required aldehyde: the overreduction of ester to alcohol that is followed by selective oxidation of alcohol to aldehyde [Dews, D. B.; Martin, J. C. (1983) “Readily accessible 12-I-5 oxidant for the conversion of primary and secondary alcohols to aldehydes and ketones” J. Org. Chem. 48, 4155; Omura, K; Swern, D. (1978) “Oxidation of alcohols by ‘activated’ dimethyl sulfoxide. A preparative, steric and mechanistic study” Tetrahedron. 34, 1651; Barriga, S. (2001) “2,2,6,6-Tetramethylpiperidine-1-oxyl (TEMPO)” Synlett 563; Montanari, F.; Quici, S.; Henry-Riyad, H.; Tidwell, T. T. (2005) “2,2,6,6-Tetramethylpiperidin-1-oxyl” Encyclopedia of Reagents for Organic Synthesis. John Wiley & Sons]. While this indirect approach is often the only option, it is far from being economic as a result of poor redox economy.

Besides non-catalytic processes, catalytic reductions of esters to aldehydes are also known. Thus, several publications describe the use of silanes as alternative reducing agents for ester substrates, together with metal and even some non-metal catalysts. A preferred silane for these types of reductions is triphenyl silane (Ph₃SiH), diethyl silane (Et₂SiH₂) or triethylsilane (Et₃SiH).

Piers et al [Parks, D. J.; Blackwell, J. M.; Piers, W. E. (2000) “Studies on the Mechanism of B(C₆F₅)₃-Catalyzed Hydrosilation of Carbonyl Functions” J. Org. Chem. 65, 3090] reported the reduction of esters to silyl acetals with Ph₃SiH and a non-metal catalyst, tris(pentafluorophenyl) borane B(C₆F₅)₃. Although the process can be used for various substrates, these reactions were accompanied with substantial overreduction (5-30%) to silyl ethers and alkanes. The Japanese patent application no. JP2016084310 (Kazumasa) describes a similar process for the reduction of α-fluorinated esters to fluorinated silyl acetals using a system composed of silane reducing agent and B, Al or Ti Lewis acids. Amongst the preferred catalysts, tris(pentafluorophenyl) borane B(C₆F₅)₃ is used in the patented process. Such a catalyst, in at least 1 mol % with respect to the substrate, is said to be appropriate for the selective and partial reduction of α-fluorinated esters to silyl acetals. Importantly, this process is limited to esters having electron withdrawing substituents in α positions.

The use of a BA₂C′ type (vide infra) triaryl borane, namely mesityl bis(perfluorophenyl) borane (Mes(F5)2 borane), as a catalyst for the partial reduction of ester functionalities is reported in Fegyverneki's doctoral dissertation [D. Fegyverneki (2018) “Szililvegyületek átalakítása triaril-boran Lewis-savakkal” doctoral dissertation, Eötvös Loránd University]. The respective method employed 5 mol % of catalyst and 1 equivalent of triethylsilane (Et₃SiH) as a reducing agent to demonstrate the capability of borane mediated hydrosilylaltion on multiple ester substrates, achieving yields between 31-99%. The resulting silyl acetals were further transformed into aldehydes resulting in yields between 50-81%. Despite the promising results, the reported Mes(F5)2 borane catalyst lacks the advantageous structural, electronic and steric properties of the novel catalysts presented in the current invention. As a result, higher catalyst loadings and longer reaction times were needed, and lower yields could be achieved compared to a process applying a catalyst according to the present invention. This was also corroborated in a comparative study, seen in Table 1: when Mes(F5)2 borane was employed under the same reaction conditions, significantly lower yield (3.4%) was detected compared to the results obtained by the catalysts presented in this invention (above 85%) (see e.g. Entry 6).

Amongst the few metal-based catalysts or catalytic systems known to perform selective reductions of esters to silyl acetals [Motoyama Y et al. (2018) “Catalytic Silane-Reduction of Carboxylic Esters and Lactones: Selective Synthetic Methods to Aldehydes, Lactols, and ethers via Silyl Acetyl Intermediates” Chemistry Select, 3, 2958; Sortais B. and Darcel C. et al (2013) “Selective Reduction of Esters to Aldehydes under the Catalysis of Well-Defined NHC-Iron Complexes” Angew. Chem. Int. Ed. 52, 8045; Wei. D and Sortais J. B. (2020) “Manganese and Rhenium-catalyzed Selective Reduction of Esters to Aldehydes with Hydrosilanes” Chem. Commun, 56,11617-11620], here should be cited the Ir catalyst-based procedure developed by Cheng and Brookhart [Cheng, C. and Brookhart, M. (2012) “Efficient Reduction of Esters to Aldehydes through Iridium-Catalyzed Hydrosilylation” Angew. Chem. Int. Ed. 51, 9422.]. Compared to other metal based catalytic systems, this system requires lower amount of catalyst, in the order of 0.1-0.5 mol %, and as a reducing agent Et₂SiH₂. However, this method uses a catalyst which is toxic and expensive, furthermore, ester substrates having olefinic functional group were not reported.

Despite reagents and processes being known in the art for partial reduction of substrates with ester or lactone functional groups, there remains a need for alternative, industrially acceptable reagents and processes for producing aldehydes or lactols from substrates containing ester groups. Especially suited for this purpose are alternative catalysts which enable processes to proceed with low catalyst loading, high conversion and high chemoselectivity for molecules containing an ester functionality and allowing the use of mild experimental conditions (temperature from approx. 25 to 50° C., ambient atmosphere, i.e. no exclusion of oxygen and humidity).

Few compounds are known which are within the scope of general formula (I) but they have different utility. These compounds are excluded by a so-called “disclaimer/proviso” part at the end of claim 1. The excluded compounds are mentioned in the following prior art documents:

In the work of Köster et al. [öster et al. (1963) “Umwandlungen bororganischer Verbindungen in der Hitze” Angew. Chem., 75, 1079-1090] the synthesis of a triaryl borane having an o-biphenylyl aryl group is disclosed. However, this compound is used only as an intermediate for the synthesis of 9-borafluorenes, without considering the use of it as a catalyst in organic reactions.

In the patent WO 2019/004172, a BA₂C type borane is presented with an o-tolyl aryl group as a substituent. This compound is subsequently used as a catalyst in the production of organoxysiloxanes by reacting siloxanes with alcohols. This reactivity however gives no clue about the potential use of the borane compound as a catalyst for the partial reduction of ester and lactone moieties.

Further, a compound having a similar structure is disclosed in the following article: Liting Li et al. (2000) “Bis(Pentafluorophenyl)(2-perfluorobiphenylyl)borane. A New Perfluoroarylborane Cocatalyst for Single-Site Olefin Polymerization” Organometallics, 19, 3332-3337, see the 2-perfluorobiphenylyl group (where one of the ortho groups relative to the boron atom is a small group (F) and the other ortho group is a large group (pentafluorophenyl). However, the compound is applied only as a catalyst in olefin polymerization and there is no hint in this article that the compound can be applied as a specific catalyst in the reduction of esters and lactones into aldehydes and lactols. Also, a similar borane, having a methyl group as the large ortho substituent, is presented in the work of Ziegler et al. [Ziegler et al. (2005) “Possible Thermal Decomposition Routes in [MeB(C₆F₅)₃]⁻[L₂TiMe⁺] as Deactivation Pathways in Olefin Polymerization Catalysis: A Combined Density Functional Theory and Molecular Mechanics Investigation” Organometallics, 24, 2076-2085], but only as a thermal decomposition side-product of B(C₆F₅)₃ catalysed olefin polymerizations. Both of these referenced studies and the respective boranes can be found in the review article Melen et al. (2020) “Halogenated triarylboranes: synthesis, properties and applications in catalysis” Chem. Soc. Rev., 49, 1706-1725. No clue is given here either for the potential use of these boranes as catalysts for the hydrosilylation of esters and lactones.

The patent CN 111574543 presents a BAA′C type (vide infra) borane, having chlorine as the large ortho substituent. However, the respective borane is used only as a starting material for the construction of larger polycyclic compounds that can be used in organic electroluminescent devices. The scope of the patent does not concern with the use of triaryl boranes as catalysts.

In the patent WO 2019/055727, two BA₂C type boranes are presented with their large ortho substituents being Cl and CF₃ groups respectively. These compounds were used as Lewis acidic polymerization catalysts for the production of polyether polyols. The document does not give any hints about their possible use for the partial reduction of esters and lactones.

In the doctoral dissertation of Bortoluzzi [J. Bortoluzzi (2018) “Biphényles a chiralitéaxiale: vers la synthese de paires de Lewis frustrées pour la catalyse énantiosélective” doctoral dissertation, Université de Strasbourg] the racemic synthesis of a BA₂C type biphenylborane is presented. However, no studies were performed on the potential application of this compound in catalytic hydrosilylation reactions.

Finally, in the article Hoshimoto et al. (2018) “Main-Group-Catalyzed Reductive Alkylation of Multiply Substituted Amines with Aldehydes Using H₂” J. Am. Chem. Soc., 140, 7292-7300, a triaryl borane is presented having a CF₃ group as the large ortho substituent. This compound was subsequently used as a catalyst for the reductive alkylation of amines, with hydrogen gas as a reducing agent. Importantly, this application does not give any clues for the borane's potential use as a catalyst for the partial reduction of esters and lactones.

As presented above, we underline that none of these documents contain any hints about the surprising effect and properties of the invented catalysts. It is important to emphasize, that the use of these compounds in a specific type of catalytic reaction does not make probable, that the catalyst can be applied in a different type of catalytic reaction as well.

### THE PROBLEM TO BE SOLVED BY THE INVENTION

The technical problem to be solved by the present invention is to provide triaryl borane type catalysts for selective hydrosilylation of esters or lactones, where the use of said catalyst in the hydrosilylation of esters or lactones has the following features:
a) low catalyst loading,
b) high conversion,
c) high chemoselectivity for molecules containing an ester functionality, especially reduction of esters of unsaturated fatty acids from natural source without any modification of the position or the stereochemistry of the olefinic double bond,
d) low overreduction of the esters and lactones to silyl ether; so low, that often no purification of the crude product is necessary,
e) mild operational conditions.

### THE DISCOVERY ACCORDING TO THE PRESENT INVENTION

During our experiments we found surprisingly that the above demands can be achieved by such triaryl borane type catalyst where the aryl groups have a special substituent pattern. Namely, in two of the aryl groups only small-size groups (e.g. H, D and F atoms) should be in the ortho positions (where the ortho positions are related to the bond connecting to the boron atom) while in the third aryl group, there should be a similar small-size group in one of the ortho positions (e.g. H, D and F atoms) and a large-size group (having larger steric demand) in the other ortho position (e.g. Cl, Br, I, SF₅, alkyl, alkenyl, cyclic alkyl, cyclic alkenyl, aryl or heteroaryl) (ortho position as defined above). The other substituents have secondary importance, but they secure the optimal Lewis acidity character of the catalyst molecule. Notably, the optimal Lewis acidity is a range, dictated by the substrates. When a more basic (oxygen Lewis basic) ester or lactone is reduced, then lower Lewis acidity is required to reach high selectivity (to suppress the overreduction), when a less Lewis basic ester (e.g. α-fluorinated, chlorinated) is reduced, higher Lewis acidity is required (to promote the Si—H bond activation).

### BRIEF DESCRIPTION OF THE INVENTION

1. Thus, in the first aspect, the invention provides compounds according to the general formula (I)

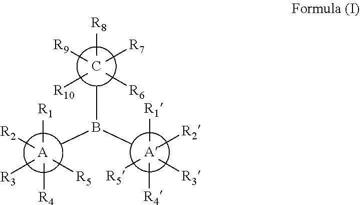

wherein
B is boron;
A ring and A′ ring, independently from each other, are aryl or heteroaryl groups, wherein
   R₁ and R′₁ are independently selected from groups having small steric demand, preferably H, D and F;
   R₅ and R′₅ are independently selected from groups having small steric demand, preferably H, D and F;
   each R₂, R₃, R₄, R′₂, R′₃ and R′₄ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
C ring is aryl group, wherein
   R₆ is selected from groups having small steric demand, preferably H, D and F;
   R₁₀ is selected from groups having large steric demand, preferably from the group consisting of Cl, Br, I, SF₅, alkyl, cycloalkyl, alkenyl, cycloalkenyl, aryl, heteroaryl and Si(R₁₅)₃ groups, where the alkyl, cycloalkyl, alkenyl, cycloalkenyl, aryl and heteroaryl groups are optionally substituted; where R₁₅ groups are selected, independently from each other, from the following scope: alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
   R₇, R₈ and R₉ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted,
   with the proviso that
   when R₁ to R₅, R′₁ to R′₅ and R₆ to R₉ are F, then R₁₀ is not pentafluorophenyl or methyl group;
   when R₁ to R₅, R′₁ to R′₅ and R₆ to R₉ are H, then R₁₀ is not phenyl;
   when R₁ to R₅, R′₁ to R′₅ are F and R₆ to R₉ are H, then R₁₀ is not methyl;
   when R₁ to R₅, R′₁, R′₃, R′₄, R′₅, R₆, R₇, R₈ are H, R′₂ and R, are Br, then R₁₀ is not Cl;
   when R₁, R₅, R_{1′}, and R_{5′} are H, R₂, R₄, R_{2′}, and R_{4′}, are CF₃, R₆ and R₈ are F, and R₇ and R₉ are H, then R₁₀ is not Cl;
   when R₁, R₅, R_{1′}, and R_{5′}, are H, R₂, R₄, R_{2′}, R_{4′}, R₆ and R₉ are CF₃, then R₁₀ is not H;
   when R₁, R₂, R₄, R₅, R_{1′}, R_{2′}, R_{4′} and R_{5′}, are F, R_{3′}, R₃, R₆, R₇ and R₈ are H, R₉ is Cl, then R₁₀ is not 2-Br-phenyl;
   when R₁, R₂, R₄, R₅, R_{1′}, R_{2′}, R_{4′} and R_{5′} are F, R_{3′}, R₃, R₆, R₇, R₈ and R₉ are H, then R₁₀ is not CF₃.

2. Another object of the invention is the use of compounds according to the general formula (I)

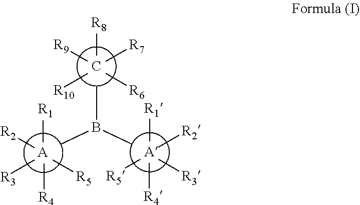

wherein
B is boron;
A ring and A′ ring, independently from each other, are aryl groups, wherein
   R₁ and R′₁ are independently selected form groups having small steric demand, preferably H, D and F;
   R₅ and R′₅ are independently selected form groups having small steric demand, preferably H, D and F;
   each R₂, R₃, R₄, R′₂, R′₃ and R′₄ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
C ring is aryl group, wherein
   R₆ is selected from groups having small steric demand, preferably H, D and F;
   R₁₀ is selected from groups having large steric demand, preferably from the group consisting of Cl, Br, I, SF₅, alkyl, cycloalkyl, alkenyl, cycloalkenyl, aryl, heteroaryl and Si(R₁₅)₃ groups, where the alkyl, cycloalkyl, alkenyl, cycloalkenyl, aryl and heteroaryl groups are optionally substituted; where R₁₅ groups are selected, independently from each other, from the following scope: alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
   R₇, R₈ and R₉ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
as a catalyst for the partial reduction of a carbonyl group in an ester substrate or lactone substrate, which substrate optionally contains one or more functional group(s) independently selected from the group consisting of non-carbonyl-conjugated olefinic bonds, non-carbonyl-conjugated acetylenic bonds, ether, amide, and halogen groups.

3. In a preferred embodiment of the present invention, the compounds of above points 1 or 2 can be characterized by general formula (Ia)

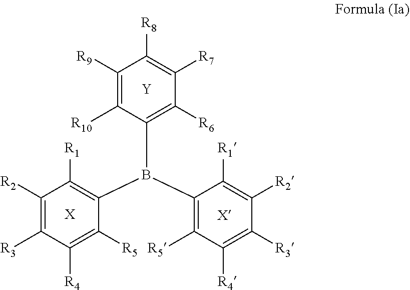

wherein
X ring and X′ ring are phenyl groups;
R₁ and R′₁ are independently selected from the group consisting of H, D and F;
R₅ and R′₅ are independently selected from the group consisting of H, D and F;
each R₂, R₃, R₄, R′₂, R′₃ and R′₄ are independently selected from the group consisting of H, D, F, Cl, Br, alkyl, cycloalkyl and aryl groups, where the alkyl, cycloalkyl and aryl groups are optionally substituted;
Y ring is phenyl group;
R₆ is selected from the group consisting of H, D and F;
R₁₀ is selected from the group consisting of Cl, Br, I, SF₅, alkyl, cycloalkyl and aryl groups, where the alkyl, cycloalkyl and aryl groups are optionally substituted;
R₇, R₈ and R₉ are independently selected from the group consisting of H, D, F, Cl, Br, alkyl and cycloalkyl groups, where the alkyl and cycloalkyl groups are optionally substituted.

4. In a further preferred embodiment of the present invention the compounds of the above points 2 and 3 have the following substituent meanings:
X ring and X′ ring are phenyl groups, wherein each R₁, R′₁, R₅ and R′₅ are F; and each R₂, R₃ R₄, R′₂, R′₃ and R′₄ are independently selected from H and F;
Y ring is phenyl group, wherein R₆ is selected from H and F; R₁₀ is selected from Cl, Br, methyl and pentafluorophenyl groups; and R₇, R₈ and R₉ are independently selected from H and F.

5. In a further preferred embodiment of the present invention the compounds of the above points 3 or 4 have the following substituent meanings:
X and X′ are independently selected from the group consisting of pentafluorophenyl, 2,3,4,6-tetrafluorophenyl, 2,3,5,6-tetrafluorophenyl, 2,4,6-trifluorophenyl, 2,3,6-trifluorophenyl and 2,6-difluorophenyl groups.

6. In a further preferred embodiment of the present invention the compounds of any one of the above points 3 to 5 have the following substituent meanings:
Y is selected from the group consisting of 2-chloro-6-fluorophenyl, 2-bromo-6-fluorophenyl, and perfluoro-1,1′-biphen-2-yl groups.

7. In a further preferred embodiment of the present invention the compounds of any one of the above points 3 to 6 are selected from following group:
(2-bromo-6-fluorophenyl)bis(2,3,5,6-tetrafluorophenyl)borane (Compound 1);
(2-bromo-6-fluorophenyl)bis(2,3,6-trifluorophenyl)borane (Compound 2);
(2-bromo-6-fluorophenyl)bis(perfluorophenyl)borane (Compound 3);
(perfluoro-[1,1′-biphenyl]-2-yl)bis(2,4,6-trifluorophenyl)borane (Compound 4);
(2-bromo-6-fluorophenyl)bis(2,4,6-trifluorophenyl)borane (Compound 5);
(2-chloro-6-fluorophenyl)bis(2,3,5,6-tetrafluorophenyl)borane (Compound 6);
perfluoro-[1,1′-biphenyl]-2-yl)bis(2,3,5,6-tetrafluorophenyl)borane (Compound 7);
perfluoro-[1,1′-biphenyl]-2-yl)bis(2,3,6-trifluorophenyl)borane (Compound 8).

8. In a more preferred embodiment of the present invention the compounds of the above point 7 are selected from the following group:
(2-bromo-6-fluorophenyl)bis(2,3,5,6-tetrafluorophenyl)borane (Compound 1);
(2-bromo-6-fluorophenyl)bis(2,3,6-trifluorophenyl)borane (Compound 2);
(2-bromo-6-fluorophenyl)bis(perfluorophenyl)borane (Compound 3);
(perfluoro-[1,1′-biphenyl]-2-yl)bis(2,4,6-trifluorophenyl)borane (Compound 4);
(2-chloro-6-fluorophenyl)bis(2,3,5,6-tetrafluorophenyl)borane (Compound 6).

9. A further object of this invention is a catalytic method for the preparation of an aldehyde or a lactol by partial reduction of a carbonyl group in an ester substrate or lactone substrate, which substrate optionally contains one or more functional group(s) independently selected from the group consisting of non-carbonyl-conjugated olefinic bonds, non-carbonyl-conjugated acetylenic bonds, ether, amide, and halogen groups, wherein the method comprises the following steps:
a) said ester or lactone substrate is reacted with a silane in the presence of a catalytic amount of a compound of formula (I) according to any one of the preceding claims to form a silyl acetal,
b) the thus-obtained silyl acetal is hydrolysed with one or more acidic or fluoride containing reagent(s) to form the aldehyde or lactol, and
c) optionally the obtained aldehyde or lactol is separated and purified.

In a preferred embodiment the functional group of the substrate is selected from the group of non-carbonyl-conjugated olefinic bond, halogen and ether functionalities.

The preferred embodiments mentioned in points 2 to 7 are preferred embodiments for the objects discussed in points 8 and 9.

10. As a further object, the present invention provides compounds according to the general formula (II)

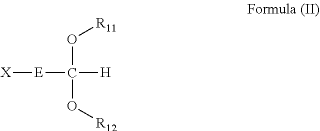

wherein
X is a halogen selected from the group consisting of Cl and Br;
E is either a (CH₂)ₘ or (CH₂)ₙ—O—(CH₂)ₚ, wherein m is an integer from 2 to 12, and n and p are, independently from each other, integers from 1 to 5, and any one of the methylene groups of (CH₂)ₘ or (CH₂)ₙ—O—(CH₂)ₚ may be optionally substituted with one or more substituent(s) [e.g. 1 to 5, or 1 to 4, or 1 to 3 or 1 or 2 substituent(s)], independently selected from each other from the group consisting of halogens, optionally substituted alkyl groups (preferably methyl or trifluoromethyl groups) or optionally substituted alkoxy groups (preferably methoxy group);
R₁₁ is a trialkylsilyl or dialkylsiloxysilyl group, where the alkyl part is an optionally substituted C₁₋₆ alkyl group, preferably C₁₋₄ alkyl group;
R₁₂ is an optionally substituted alkyl group, preferably C₁₋₆ alkyl group, preferably C₁₋₃ alkyl group.

11. In a preferred embodiment of the present invention, the compounds of the above point 10 have the following substituent meanings:
X is a halogen selected from the group consisting of Cl and Br;
E is either a (CH₂)ₘ or (CH₂)ₙ—O—(CH₂)ₚ, wherein m is an integer from 2 to 10, and n and p are, independently from each other, integers from 1 to 3, and any one of the methylene groups of (CH₂)ₘ or (CH₂)ₙ—O—(CH₂)ₚ may be optionally substituted with 1 to 3 substituent(s) [e.g. 1 or 2 substituent(s)], independently selected from each other from the group consisting of halogens, optionally substituted alkyl groups (preferably methyl group) or optionally substituted alkoxy groups (preferably methoxy group);
R₁₁ is a trialkylsilyl or dialkylsiloxysilyl group, where the alkyl part is a C₁₋₂ alkyl group, preferably triethylsilyl group;
R₁₂ is a C₁₋₃ alkyl group, preferably methyl, ethyl, propyl or isopropyl group.

12. In a further preferred embodiment of the present invention the compounds of the above point 10 or point 11 are selected from the following group:
(4-bromo-1-ethoxybutoxy)triethylsilane (Example 14)
(3-bromo-1-ethoxypropoxy)triethylsilane (Example 15)
((5-bromo-1-ethoxypentyl)oxy)triethylsilane (Example 16)
((6-bromo-1-ethoxyhexyl)oxy)triethylsilane (Example 17)
(4-bromo-1-isopropoxybutoxy)triethylsilane (Example 18)
(2-(2-chloroethoxy)-1-ethoxyethoxy)triethylsilane (Example 19)
(2-(2-bromoethoxy)-1-ethoxyethoxy)triethylsilane (Example 21)
(4-bromo-1-ethoxy-2-fluorobutoxy)triethylsilane (Example 22)
((4-bromo-1-ethoxypentyl)oxy)triethylsilane (Example 24)
(4-bromo-1-ethoxy-2,2-difluorobutoxy)triethylsilane (Example 25)
(4-bromo-1-ethoxy-2-methylbutoxy)triethylsilane (Example 26).

13. As a further object, the present invention provides compounds according to the general formula (III)

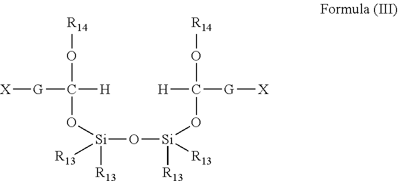

wherein
X is a halogen selected from the group consisting of Cl and Br;
G is either a (CH₂)ₘ or (CH₂)ₙ—O—(CH₂)ₚ, wherein m is an integer from 2 to 12, and n and p are, independently from each other, integers from 1 to 5, and any one of the methylene groups of (CH₂)ₘ or (CH₂)ₙ—O—(CH₂)ₚ may be optionally substituted with one or more substituent(s) [e.g. 1 to 5, or 1 to 4, or 1 to 3 or 1 or 2 substituent(s)], independently selected from each other from the group consisting of halogens, optionally substituted alkyl groups (preferably methyl or trifluoromethyl groups) or optionally substituted alkoxy groups (preferably methoxy group);
R₁₃ is an optionally substituted alkyl group, preferably a C₁₋₆ alkyl group, more preferably methyl group;
R₁₄ is an optionally substituted alkyl group, preferably a C₁₋₆ alkyl group, more preferably C₁₋₃ alkyl group.

14. In a preferred embodiment of the present invention, the compounds of the above point 13 have the following substituent meanings:
X is a halogen selected from the group consisting of Cl and Br;
G is either a (CH₂)ₘ or (CH₂)ₙ—O—(CH₂)ₚ, wherein m is an integer from 2 to 10, and n and p are, independently from each other, integers from 1 to 3, and any one of the methylene groups of (CH₂)ₘ or (CH₂)ₙ—O—(CH₂)ₚ may be optionally substituted with 1 to 3 substituent(s) [e.g. 1 or 2 substituent(s)], independently selected from each other from the group consisting of halogens, optionally substituted alkyl groups (preferably methyl group) or optionally substituted alkoxy groups (preferably methoxy group);
R₁₃ is an optionally substituted alkyl group, preferably
C₁₋₃ alkyl group, more preferably methyl group;
R₁₄ is an optionally substituted alkyl group, preferably C₁₋₃ alkyl group, more preferably methyl, ethyl, propyl or isopropyl group.

15. In a further preferred embodiment of the present invention the compound of the above point 13 or point 14 is 4,10-bis(3-bromopropyl)-6,6,8,8-tetramethyl-3,5,7,9,11-pentaoxa-6,8-disilatridecane (Example 27).

### DETAILED DESCRIPTION OF THE INVENTION

During our research, we investigated the hydrosilylation reactions of esters using different Frustrated Lewis-pair (FLP) based borane catalysts (Stephan, D. W.; Erker, G. (2015) “Frustrated Lewis Pair Chemistry: Development and Perspectives” Angew. Chem. Int. Ed., 54, 6400). Methyl 3-phenylpropionate was chosen as a model compound, the reduction of which was carried out with triethylsilane (TESH, a preferred silane compound) according to the following reaction scheme:

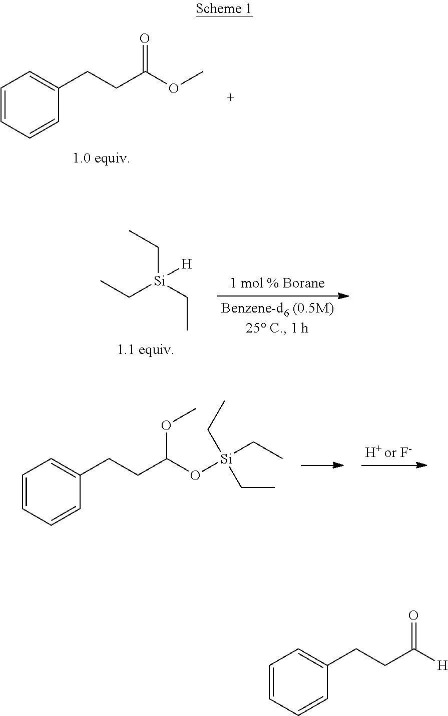

From the point of view of the applicability of the method, it is important to mention the most significant side reaction, which is the overreduction of the formed silyl acetal to silyl ether (from which the relating alcohol is formed by hydrolysis) according to the following reaction scheme:

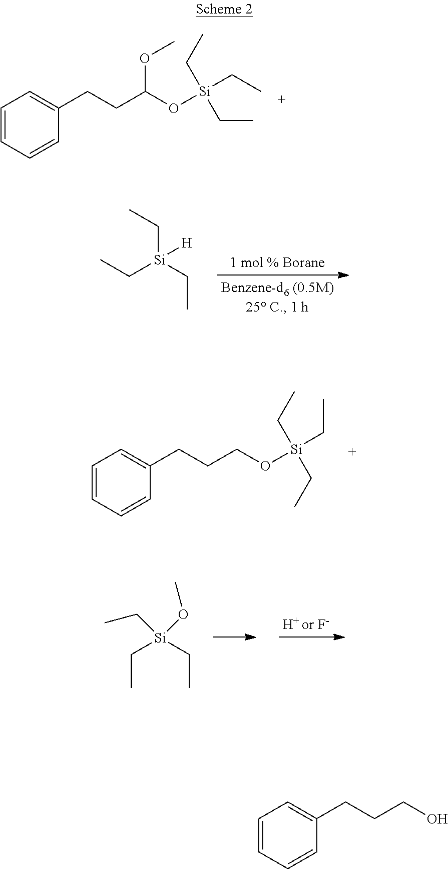

Suppression of this side reaction is almost as important as achieving the high conversion and yield. This can be especially important in certain pheromone syntheses, as the purification of the alcohol side product is cumbersome for long-chain, unsaturated fatty acid derived sex pheromone aldehydes. The alcohol impurities are relevant, as most of them act as a behavioral antagonist, e.g. Xu et al (2016): “Olfactory perception and behavioral effects of sex pheromone gland components in Helicoverpa armigera and Helicoverpa assulta” Sci. Rep. 6, 22998. Thus, the alcohol content in the reaction product cannot exceed a certain level in the final product in pheromone applications. Therefore, the suppression of overreduction (preferably having almost exclusive selectivity for silyl acetal formation) is a key technological feature in economically important application areas.

The present invention is based on the surprising fact that the use of electronically capable and specially functionalized borane catalyst having special electronic and steric properties considerably enhanced the reactivity and selectivity in the hydrosilylation of esters and lactones. The advantageous electronic and steric properties are the results of a special substituent pattern, wherein in a BAA′C type borane the A and A′ aryl (preferably phenyl) groups have only small-size groups (e.g. H, D and F atoms) in the ortho positions while in the third aryl group (C, preferably phenyl) there should be a similar small-size group in one of the ortho positions (e.g. H, D and F atoms) and a large-size group (having larger steric demand) in the other ortho position (e.g. Cl, Br, I, SF₅, alkyl, alkenyl, cyclic alkyl, cyclic alkenyl group, aryl, halogenated aryl (preferably trifluoro-, tetrafluoro- or pentafluoro (i.e. perfluoro-) phenyl, more preferably perfluorophenyl) or heteroaryl group, preferably Cl, Br, I, trifluoro-, tetrafluoro- or perfluorophenyl or methyl groups, more preferably Br) (ortho position as defined above). The large group can also be a Si(R₁₅)₃ group, where the R₁₅ groups are selected, independently from each other, from the following scope: alkyl, cycloalkyl, alkenyl, cycloalkenyl, aryl and heteroaryl groups, where the alkyl group is preferred, especially the methyl group.

Here we mention that, theoretically, the —OCF₃ group could also behave as a small-sized group without weakening the acidic character of the borane owing to the electron withdrawing effect of the fluorine atoms.

As it was mentioned above, the other substituents are of secondary importance, but they should ensure the necessary Lewis acidic character to the boron atom. For this reason, most of them should be electron withdrawing groups, e.g. F and/or Cl atoms. If not all the groups are electron withdrawing groups (which also may happen, see the perfluorinated rings), then the remaining substituents can be selected e.g. from the group consisting of H, D, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted, preferably H, D, alkyl and aryl, more preferably H and alkyl, e.g. H.

In advantageous embodiments A and A′ aryl groups are equivalent groups, i.e. having the same substitution pattern (these compounds can be signed as BA₂C type boranes). The synthesis of these symmetric molecules is much simpler (since the same reagent can be used for the formation of two rings). But it can be solved by a skilled person that the A and A′ aryl groups have different substituent patterns while having practically the same electron withdrawing character and steric parameters in ortho positions, so the substituent definition should embrace these obvious equivalents, too.

Here we mention that we found in our experiments that BA₂C′ type boranes having C′ aryl group with two large-size groups with large steric demand (e.g. Cl, Br or methyl, see Entry 4 and 9 below) in R₆ and R₁₀ ortho positions or BAC, type substitution pattern show low activity and/or low selectivity when used for the reduction of ester and lactone compounds. As described in the prior art, the system B(C₆F₅)₃ (BA₃ type catalyst)—having only small-size F substituents in ortho positions—is not capable of reducing a usual ester or lactone with such a selectivity (see Entry 1 below), in contrast to the reductive system of the present invention applying the invented catalysts.

During our experiments we prepared a remarkable number of catalyst compounds in accordance with the processes described in the Examples part. These processes are based on synthetic procedures known in the art for the preparation of BA₂C type boranes. First, a boronic acid intermediate is constructed that bears the C aryl group of the final borane. Then, this boronic acid is in turn converted to its respective potassium trifluoroborate salt using potassium hydrogenfluoride as a fluoride source. This reaction is generally carried out in water-methanol solvent mixtures, at ambient temperature and pressure. The obtained trifluoroborate salts are much more stable compared to their boronic acid precursors, i.e. they have longer shelf lives and higher air and moisture stabilities. Also, they have the necessary reactivity for the next synthetic step, that involves reacting the trifluoroborate salt in an ethereal solvent (e.g. diethyl ether or tetrahydrofuran, preferably diethyl ether) with two equivalents of an aryl Grignard reagent bearing the A aryl group to form the respective BA₂C borane. The reaction temperature can vary within a wide range of values, and will in general be in the range of −78° C. to 40° C., preferably between 0° C. and 30° C. The pressure applied in these reactions is atmospheric in general. The needed Grignard reagents can be prepared from the respective aromatic compounds by a number of procedures know in the art, e.g. by reacting the respective aryl halide directly with magnesium metal, by reacting the aryl halide with a transfer Grignard reagent (e.g. isopropylmagnesium chloride) to conduct a halogen-magnesium exchange, or by deprotonating the respective aromatic compound using an organolithium reagent (e.g. n-butyllithium) and trans-metalation with magnesium bromide to form a Grignard reagent. The final step of the borane synthesis is the purification procedure, that involves a solvent exchange to toluene, inert filtration of the precipitates, in vacuo evaporation of the toluene filtrate, sonication of the obtained residue in pentane or hexanes and inert filtration of the resulting suspension to obtain the borane as a crystalline powder.

As seen in the following Table 1, we tested the effectiveness of the prepared catalyst compounds in a reaction where methyl 3-phenylpropionate was applied as ester substrate (see the details below). As it can be seen, Entries 1 to 9, 13, and 14 were not effective (low conversion or high conversion with wrong selectivity, i.e. with a remarkable overreduction) because they did not have the necessary substituent pattern. However, Entries 10, 11, 12, 15, 16 and 17 having the substituent pattern according to the present invention, showed an excellent conversion rate and yield, together with good/acceptable contamination profile.

Here we mention that Entries 19 and 20 did not show really good results in this test reaction (the yield was low), but they can be applied with success in such reductions, where a catalyst with weaker Lewis acidic character is needed (see Example 12, where Compound 5 (Entry 19) was applied as a catalyst with very good results in the reduction of a more Lewis basic lactone (namely γ-Butyrolactone), or Example 28, where using Compound 9 (Entry 20) as a catalyst, the selectivity of the reduction was conserved even while using a more reactive silane as a reducing agent (namely 1,1,3,3-tetramethyldisiloxane)).

Moreover, Entry 18 showed poor results in this test reaction (the selectivity was low), but it can be applied with success in such reductions, where a catalyst with stronger Lewis acidic character is needed (see Example 11, where this catalyst compound was applied with very good results in reduction of an weakly Lewis basic ester type substrate, namely ethyl 2,2,2-trifluoroacetate).

These examples prove a further advantage of the catalyst compounds according to the invention since their Lewis acidity can be adjusted to the necessary level, matching it to the Lewis basic character of the substrate (on the basis of the general knowledge a skilled person who knows which substituents increase and which decrease the Lewis acidic/basic character of the catalyst and the substrate, respectively).

The test reactions in Table 1. were monitored by quantitative NMR measurements using hexamethylbenzene (HMB) as internal standard. Notably, a small excess of the reducing agent, e.g. TESH (1.1 equiv.) was used to secure higher or full conversions and to reveal in the test reactions, whether the investigated catalytic system is prone to overreducing the substrate. Also, the employed catalyst loads were higher than needed (1 mol %) to secure shorter reaction times and to illustrate the differences in selectivity. Using the obtained spectra, we successfully determined the composition of the reaction mixtures after 1 hour of reaction time (4 hours in the case of Entry 16) for the following components: methyl 3-phenylpropionate (ester, starting material, for calculation of conversion), triethylsilane (TESH, starting material), triethyl-(1-methoxy-3-phenylpropoxy) silane (silyl acetal, main product, for calculation of product yield), triethyl-(3-phenylpropoxy) silane (silyl ether, overreduction side-product, for monitoring the selectivity) and triethylmethoxysilane (TESOMe, overreduction side-product, for monitoring the selectivity). TESOMe and silyl ether are formed in the same reaction step, therefore, their amounts should be the same. Nevertheless, the silyl ether may be involved in further reactions, thus, monitoring these two components may also provide additional information on the selectivity/overreduction of the reaction.

TABLE 1

| | | | | | Product | Other Components (%) | | |
|---|---|---|---|---|---|---|---|---|
| | | | | Conversion | Yield | Silyl | | |
| Entry | Code | Structure | Name | (%) | (%) | ether | TESOMe | TESH |
| 1 | (F5)3 borane | 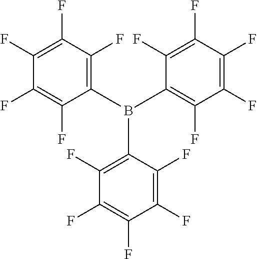 [SMILES: Fc1c(F)c(F)c(B(c2c(F)c(F)c(F)c(F)c2F)c2c(F)c(F)c(F)c(F)c2F)c(F)c1F] | tris(perfluoro- phenyl)borane | 90.3 | 59.3 | 9.3 | 12.3 | 0.0 |
| 2 | (F4)3 borane | 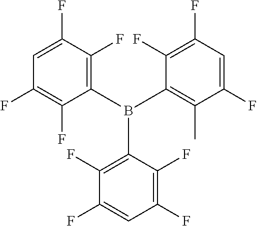 [SMILES: Cc1c(F)cc(F)c(F)c1B(c1c(F)c(F)cc(F)c1F)c1c(F)c(F)cc(F)c1F] | tris(2,3,5,6- tetrafluoro- phenyl)borane | 100.0 | 82.1 | 2.7 | 7.5 | 0.0 |
| 3 | F2(F5)2 borane | 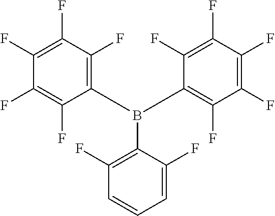 [SMILES: Fc1cccc(F)c1B(c1c(F)c(F)c(F)c(F)c1F)c1c(F)c(F)c(F)c(F)c1F] | (2,6-difluoro- phenyl)bis- (perfluoro- phenyl)borane | 93.0 | 70.3 | 11.6 | 11.7 | 0.0 |
| 4 | Cl2(F5)2 borane | 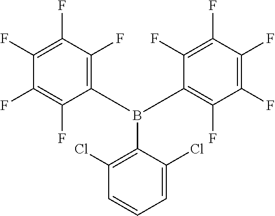 [SMILES: Fc1c(F)c(F)c(B(c2c(Cl)cccc2Cl)c2c(F)c(F)c(F)c(F)c2F)c(F)c1F] | (2,6-dichloro- phenyl)bis- (perfluoro- phenyl)borane | 82.9 | 69.6 | 6.5 | 6.0 | 14.1 |
| 5 | Br2(F5)2 borane | 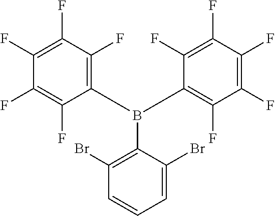 [SMILES: Fc1c(F)c(F)c(B(c2c(Br)cccc2Br)c2c(F)c(F)c(F)c(F)c2F)c(F)c1F] | (2,6-dibromo- phenyl)bis- (perfluoro- phenyl)borane | 15.8 | 8.2 | 0.4 | 0.5 | 73.1 |
| 6 | Mes(F5)2 borane | 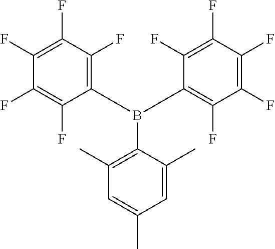 [SMILES: Cc1cc(C)c(B(c2c(F)c(F)c(F)c(F)c2F)c2c(F)c(F)c(F)c(F)c2F)c(C)c1] | mesitylbis- (perfluoro- phenyl)borane | 9.9 | 3.4 | 0.0 | 0.0 | 76.0 |
| 7 | Cl2Mes(F5)2 borane | 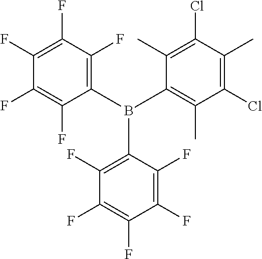 [SMILES: Cc1c(Cl)c(C)c(B(c2c(F)c(F)c(F)c(F)c2F)c2c(F)c(F)c(F)c(F)c2F)c(C)c1Cl] | (3,5-dichloro- 2,4,6-trimeth- ylphenyl)bis- (perfluoro- phenyl)borane | 10.7 | 0.2 | 0.0 | 0.0 | 74.7 |
| 8 | Mes(F4)2 borane | 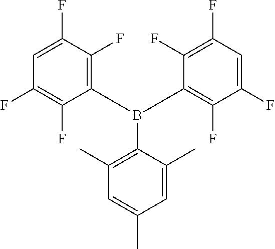 [SMILES: Cc1cc(C)c(B(c2c(F)c(F)cc(F)c2F)c2c(F)c(F)cc(F)c2F)c(C)c1] | mesitylbis- (2,3,5,6-tetra- fluorophenyl) borane | 5.8 | 0.4 | 0.0 | 0.0 | 79.9 |
| 9 | Mes(F3a)2 borane | 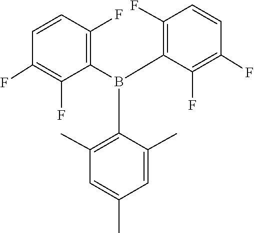 [SMILES: Cc1cc(C)c(B(c2c(F)ccc(F)c2F)c2c(F)ccc(F)c2F)c(C)c1] | mesitylbis- (2,3,6- trifluorophen- yl)borane | 6.5 | 1.0 | 0.0 | 0.0 | 81.6 |
| 10 | ClF(F4)2 borane | 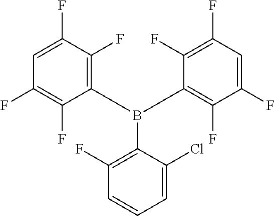 [SMILES: Fc1cc(F)c(F)c(B(c2c(F)cccc2Cl)c2c(F)c(F)cc(F)c2F)c1F] | (2-chloro-6- fluorophen- yl)bis(2,3,5,6- tetrafluoro- phenyl)borane | 99.9+ | 87.8 | 1.1 | 3.0 | 5.0 |
| 11 | BrF(F5)2 borane | 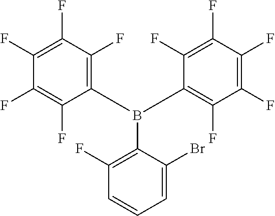 [SMILES: Fc1cccc(Br)c1B(c1c(F)c(F)c(F)c(F)c1F)c1c(F)c(F)c(F)c(F)c1F] | (2-bromo-6- fluorophen- yl)bis(per- fluorophenyl) borane | 99.9+ | 84.2 | 4.2 | 5.2 | 0.4 |
| 12 | BrF(F4)2 borane | 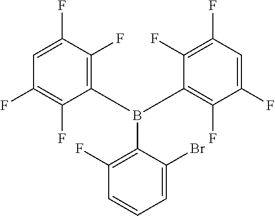 [SMILES: Fc1cc(F)c(F)c(B(c2c(F)cccc2Br)c2c(F)c(F)cc(F)c2F)c1F] | (2-bromo-6- fluorophenyl) bis(2,3,5,6- tetrafluoro- phenyl)borane | 99.9+ | 88.6 | 0.5 | 0.6 | 7.1 |
| 13 | (F9)3 borane | 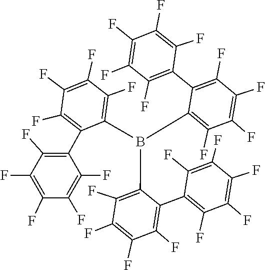 [SMILES: Fc1c(F)c(F)c(-c2c(F)c(F)c(F)c(F)c2B(c2c(F)c(F)c(F)c(F)c2-c2c(F)c(F)c(F)c(F)c2F)c2c(F)c(F)c(F)c(F)c2-c2c(F)c(F)c(F)c(F)c2F)c(F)c1F] | tris(perfluoro- [1,1′-biphen- yl]-2-yl)borane | 19.9 | 0.0 | 13.1 | 12.3 | 55.5 |
| 14 | Cl2(F9)2 borane | 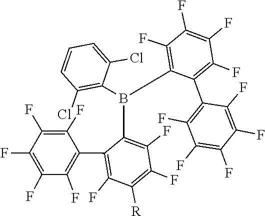 | (2,6-dichloro- phenyl)bis- (perfluoro- [1,1′-biphen- yl]-2-yl)borane | 11.5 | 0.0 | 0.0 | 0.0 | 75.4 |
| 15 | F9(F3s)2 borane | 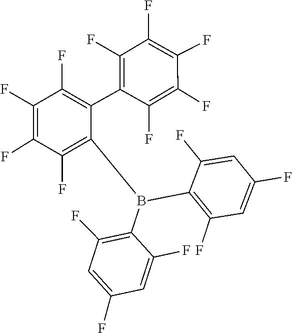 [SMILES: Fc1cc(F)c(B(c2c(F)cc(F)cc2F)c2c(F)c(F)c(F)c(F)c2-c2c(F)c(F)c(F)c(F)c2F)c(F)c1] | (perfluoro- [1,1′-biphen- yl]-2-yl)bis- (2,4,6-trifluoro- phenyl)borane | 99.9+ | 92.8 | 0.1 | 0.1 | 6.2 |
| 16 | BrF(F3a)2 borane | 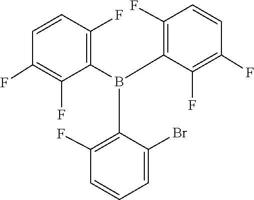 [SMILES: Fc1ccc(F)c(B(c2c(F)cccc2Br)c2c(F)ccc(F)c2F)c1F] | (2-bromo-6- fluorophenyl)- bis(2,3,6-tri- fluorophenyl) borane | 94.2 | 90.5 | 0.0 | 0.1 | 12.3 |
| 17 | F9(F3a)2 borane | 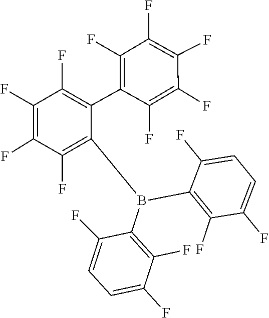 [SMILES: Fc1ccc(F)c(B(c2c(F)ccc(F)c2F)c2c(F)c(F)c(F)c(F)c2-c2c(F)c(F)c(F)c(F)c2F)c1F] | (perfluoro- [1,1′-biphen- yl]-2-yl)bis- (2,3,6-trifluoro- phenyl)borane | 99.9+ | 94.6 | 4.3 | 4.2 | 3.6 |
| 18 | F9(F4)2 borane | 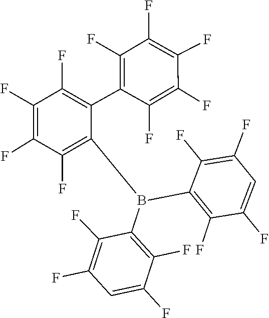 [SMILES: Fc1cc(F)c(F)c(B(c2c(F)c(F)cc(F)c2F)c2c(F)c(F)c(F)c(F)c2-c2c(F)c(F)c(F)c(F)c2F)c1F] | (perfluoro- [1,1′-biphen- yl]-2-yl)bis- (2,3,5,6-tetra- fluorophenyl) borane | 99.9+ | 80.1 | 11.2 | 11.1 | 0.00 |
| 19 | BrF(F3s)2 borane | 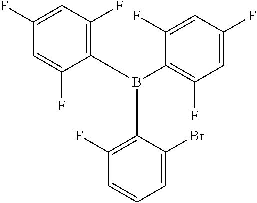 [SMILES: Fc1cc(F)c(B(c2c(F)cc(F)cc2F)c2c(F)cccc2Br)c(F)c1] | (2-bromo-6- fluorophen- yl)bis(2,4,6- trifluorophen- yl)borane | 11.4 | 5.0 | 0.0 | 0.0 | 73.1 |
| 20 | BrF(F2)2 borane | 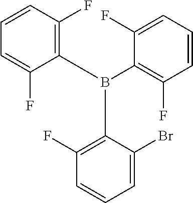 [SMILES: Fc1cccc(F)c1B(c1c(F)cccc1F)c1c(F)cccc1Br] | (2-bromo-6- fluorophen- yl)bis(2,6- difluorophen- yl)borane | 1.8 | 1.1 | 0.00 | 0.00 | 79.0 |
| 21 | CH3(F5)2 borane | 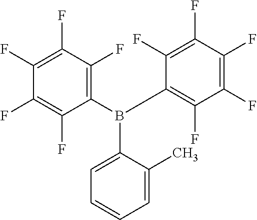 [SMILES: Cc1ccccc1B(c1c(F)c(F)c(F)c(F)c1F)c1c(F)c(F)c(F)c(F)c1F] | (o-tolyl) bis- (perfluoro- phenyl)borane | 93.3 | 91.1 | 1.4 | 1.4 | 11.2 |

As used herein, D is deuterium, which is an isotope of hydrogen (H), having the same chemical properties as H, so it can replace H without changing the chemical character of the molecule. Obviously, D is also a “group having small steric demand”.

As used herein, the term “alkyl” alone or in combinations means a linear (straight) or branched-chain alkyl group containing from 1 to 20, preferably 1 to 8, more preferably 1 to 6 or 1 to 5 carbon atom(s) (i.e. “C₁₋₆” or “C₁₋₅” alkyl groups), such as methyl, ethyl, propyl, isopropyl, butyl, sec-butyl, t-butyl and pentyl groups. In special cases this phrase can relate to alkyl groups containing from 1 to 4, or 1 to 3 or 1 to 2 carbon atom(s) (i.e. “C₁₋₄” or “C₁₋₃” or “C₁₋₂” alkyl groups), where the methyl is a preferred embodiment.

As used herein, the term “cycloalkyl” means a group that is derived from a C₃₋₈, preferably C₃₋₆ cycloalkane by removal of a hydrogen atom from the ring, for example cyclopropyl, cyclobutyl, cyclopentyl and cyclohexyl groups.

As used herein, the term “alkenyl” means an aliphatic hydrocarbon group containing at least one carbon-carbon double bond and which may be linear (straight) or branched and comprising 2 to 20, preferably 2 to 10, more preferably 2 to 6 carbon atoms in the chain.

Branched means that one or more lower alkyl groups such as methyl, ethyl or propyl, are attached to a linear alkenyl chain. Non-limiting examples of suitable alkenyl groups include ethenyl (vinyl), propenyl, n-butenyl, 3-methylbut-2-enyl and n-pentenyl groups.

As used herein, the term “cycloalkenyl” means a C₃₋₈, preferably C₄₋₆ cyclic hydrocarbon group containing at least one carbon-carbon double bond (preferably one double bond), for example cyclobutenyl or cyclopentenyl groups.

As used herein the term “aryl”, alone or in combinations means a group derived from an aromatic monocyclic or polycyclic ring system comprising 6 to 14 carbon atoms, preferably 6 to 10 carbon atoms, more preferably 6 carbon atoms, e.g. phenyl, naphthyl or azulenyl, especially phenyl groups.

As used herein, the term “heteroaryl” means a group derived from a monocyclic or bicyclic aromatic ring system (condensed double ring systems) with 1 to 3 heteroatom(s) selected from the group consisting of N, O and S [i.e. group of N (nitrogen), O (oxygen) or S (sulfur) atoms], where the other ring forming atoms are carbon atoms. In a preferred embodiment the “heteroaryl” means a group derived from a bicyclic aromatic ring system with 1 to 2 heteroatom(s) selected from the group consisting of O and S and the other ring forming atoms are carbon atoms, see e.g. benzofuran and thiophene.

The above-mentioned alkyl, cycloalkyl, alkenyl, cycloalkenyl, aryl and heteroaryl groups may be optionally substituted with one or more substituent(s) [e.g. 1 to 5, or 1 to 4, or 1 to 3 or 1 or 2 substituent(s), independently selected from each other] usually applied in the organic chemistry for substitution of such groups. So, the substituted groups carry one or more, preferably one to three substituent(s), independently selected from the group consisting of halogen, optionally substituted alkyl (more preferably methyl and trifluoromethyl), optionally substituted alkoxy (more preferably methoxy), hydroxyl, alkoxy, haloalkyl, sulphate, amino, amide, acylamino, monoalkylamino, dialkylamino, alkylthio, alkylsulfinyl, alkylsulfonyl groups, where alkyl (more preferably methyl and trifluoromethyl), halogen, hydroxyl, alkoxy (more preferably methoxy, optionally substituted with halogen, e.g. fluoro), especially halogen, alkyl and alkoxy, e.g. alkyl and alkoxy optionally substituted with halogen are more specific examples.

For the purposes of the present invention, the term “substrate” shall mean an ester or lactone (which can be regarded as a cyclic ester) to be subjected to a reaction with a silane in the presence of a catalyst to obtain silyl acetal. Said substrate includes saturated or unsaturated esters or lactones. Non-limiting examples for saturated and unsaturated esters are as follows: acetates, trifluoroacetates, propionates, butyrates, isobutyrates, benzoates, dihydrocinnamates, cis-3-hexenoates, 10-undecylenates, 11-eicosenoates, alpha-eleostearates, oleates, linoleates, esters of natural saturated and unsaturated fatty acids, e.g. pheromone precursors and mixtures thereof. All the above-cited esters may, for example, be alkyl or phenolic esters, e.g. C₁-C₂₂, preferably C₁₆-C₂₀ or C₁₋₆ or C₁₋₄ or C₁₋₂ alkyl esters (preferably methyl and ethyl esters, see e.g. ethyl acetate, methyl butyrate etc), which are optionally substituted, e.g. by aryl, preferably by phenyl (see e.g. 3-phenylpropionate esters, preferably methyl 3-phenylpropionate). Non-limiting examples for saturated and unsaturated lactones are as follows: butyrolactone, valerolactone, caprolactone, decalactone, dodecalactone.

As used in the present invention, the term “silyl acetal” means a mixed acetal that results from the hydrosilylation of the ester or lactone substrates. The formed mixed acetal consists of a siloxy group, resulting from the silylation of the substrate's carbonyl group with the respective silane; and of an alkoxy group that originates from the alkoxy group of the substrate's ester or lactone moiety.

As laid out above, the reduction according to the invention is applicable to various esters and lactone compounds which may contain different functions, like unsaturated bonds [one or more non-carbonyl-conjugated olefinic double bond and/or acetylenic triple bond], alkyl or aryl ethers, amides and halogen group(s), which will not be affected by the reduction reaction.

A remarkable property of the catalysts according to the invention is that they allow the reduction of natural triglycerides of fatty acids [e.g. saturated or unsaturated fatty acids having 12 to 24 carbon atoms, preferably 16 to 22 carbon atoms and, in another preferred embodiment, 1 to 5, preferably 1 to 3 double bond(s)], like those which form the vegetable (e.g. tung oil) and animal oils. In the course of the reaction of a mixed triglyceride derived from distinct fatty acids, there can be obtained simultaneously saturated and unsaturated natural aldehydes without any modifications of the position or of the stereochemistry of the olefinic double bonds. This is of particular value for olefinic bonds having a cis-configuration.

In the case where these substrates contain one or more olefinic groups with defined stereochemistry (which, in general, will be cis), the corresponding acetal obtained after reduction according to the invention will have the same stereochemistry. Thus, oils rich in linoleic and/or linolenic acid, like linseed oil, will be transformed into mixtures rich in linoleyl and/or linolenyl aldehyde.

Other oils and fats which are found in nature and which are not triglycerides, but esters of unsaturated fatty acids and monovalent unsaturated alcohols [where the chains deriving from the fatty acid and the alcohol have, independently from each other, 12 to 24 carbon atoms, preferably 16 to 22 carbon atoms and, in another preferred embodiment, 1 to 5, preferably 1 to 3 double bond(s)], like jojoba oil and sperm oil, can also be reduced according to the present invention, without any modification of the position or of the stereochemistry of the double bonds present in the ester molecules.

A great number of silanes can be used in the process according to the present invention. Such silanes are known to a person skilled in the art, and they will be chosen according to their capacity to effectively reduce ester or lactone substrates in the process according to the present invention. As non-limiting examples, there can be cited trialkylsilanes (e.g. triethylsilane), alkoxydialkylsilanes, dialkoxyalkylsilanes, trialkoxysilanes (e.g. trimethoxysilane), dialkylsilanes (e.g. diethylsilane), alkylsilanes or triarylsilanes, diarylsilanes, arylsilanes (e.g. phenylsilane), diarylalkylsilanes, aryldialkylsilanes (e.g. dimethylphenylsilane), arylalkylsilanes (e.g. methylphenylsilane), trisiloxysilanes, alkyldisiloxysilanes, dialkylsiloxysilanes (e.g. 1,1,3,3-tetramethyldisiloxane (TMDS)) or poly(alkylhydrosiloxane) polymers [preferably poly(methylhydrosiloxane) polymers (PMHS)], where the siloxy group is an alkylsiloxy or dialkylsiloxy group, preferably dimethylsiloxy group, and where the alkyl part contains 1 to 6 carbon atoms, preferably 1 to 4 carbon atoms, the aryl group is phenyl or naphthyl group, preferably phenyl group. In a preferred embodiment of the present invention said silane is triethylsilane (TESH) or 1,1,3,3-tetramethyldisiloxane (TMDS) due to their effectiveness, availability, and price.

The concentration of the catalyst according to the present invention, given in mol % with respect to the substrate, is generally from 0.005 to 2.0% by mole, preferably 0.01 to 1.0% by mole, more preferably from 0.03 to 0.2%. Low catalyst levels are preferred because these reduce the overall costs of catalytic partial reductions.

There will typically be consumed 1.0 mol equivalents of silane compound (e.g. TESH) per 1.0 mol of ester or lactone function. For practical reasons, there will preferably be used a slight excess of silane compound with respect to these stoichiometric amounts, in general of the order of 1 to 15 mol % excess, preferably 2 to 5 mol % excess, based on the stoichiometric quantity. The reduction reaction according to the invention also takes place when the silane is used in sub-stoichiometric amounts, but this results in a decrease of conversion.

The selectivity of the reaction even enables the use of larger excess of silane (up to 2 equiv. or more) if quicker reactions are needed. However, in these cases, overreduction is possible when the necessary reaction times are significantly extended (5-10 times).

The reduction can be carried in a solvent such as, for example, an ether (e.g. methyl-tetrahydrofuran, diethyl ether, methyl tert-butyl ether, diisopropyl ether, dibutyl ether, tert-amyl methyl ether, tetrahydrofuran or dioxane), an aliphatic hydrocarbon (e.g. hexane, heptane, petroleum ether, octane, or cyclohexane) or an aromatic hydrocarbon (e.g. benzene, toluene, xylene or mesitylene), or mixture thereof. Low levels of solvent, or even solvent-free systems may be employed. Low levels of solvent include <100% solvent per substrate in weight equivalents (m/m), <50% m/m, <25% m/m or preferably <10% m/m. Deuterated solvent can be also applied, like benzene-d6.

The reaction temperature can vary within a wide range of values, and will in general be in the range of −20° C. to 60° C. The temperature chosen will depend on the reactivity of the substrate and can be adjusted accordingly without difficulty. Preferably, the reaction is conducted at a temperature within the range of 20 to 60° C., preferably 30 to 45° C.

The pressure applied in the reactions is atmospheric in general. However, elevated pressure (e.g. 2 to 10 atm) can be useful, especially if one of the components is a gas or a highly volatile compound.

The order of the addition of the reactants is also interchangeable. Premixing either two of the components (substrate, catalyst and silane compound) and dropwise addition of the third reactant is possible.

The respective aldehydes or lactols can be obtained by acidic or F⁻ (fluoride) induced hydrolysis of the formed silyl acetal. This hydrolysis is known in the art and may be carried out by adding to the reaction mixture an aqueous or alcoholic solution (or a solution made from a mixture of water and an organic solvent e.g. acetonitrile, THF) of an acidic reagent such as, for example, acetic acid, HCl, sulphuric acid or even silica gel, or a fluoride containing reagent e.g. aq. TBAF, H₂SiF₆. The ratio of the hydrolysing reagents with respect to the silane compound (e.g. TESH) used will be from about 0.01 to 0.1 mol equivalents. After complete hydrolysis, generally, formation of two phases is observed. The desired aldehyde is typically found in the organic phase and can be obtained by evaporation of the solvent which may be present. The obtained residue may be distilled, partitioned between two phases (e.g. hexanes/CH₃CN), chromatographed or steam distilled for further purification (carried out in line with the general knowledge of a skilled person), if needed.

The hydrolysis is conducted preferably at a temperature within the range of 0 to 100° C., more preferably 10 to 45° C., even more preferably ambient temperature. The pressure applied in the reaction is atmospheric in general.

The invention will now be illustrated in greater detail in the following examples in which the temperatures are indicated in degrees centigrade, the yields in mol %, the chemical shift of the NMR data in ppm, relative to tetramethylsilane as internal reference, and the abbreviations have the usual meaning in the art.

### EXAMPLES

In Examples 1 to 8 and Example 26, the preparation of those compounds of formula (I) are disclosed which are given in Table 1. The other (reference) compounds of Table 1 were synthetized by analogous processes or were obtained from commercial sources.

### Example 1

### Synthesis of (2-bromo-6-fluorophenyl)bis(2,3,5,6-tetrafluorophenyl)borane (Compound 1, see Entry 12)

The compound was prepared as described below and illustrated in schemes 3 to 5.

Step a) Synthesis of (2-bromo-6-fluorophenyl)boronic acid (Compound 1a)

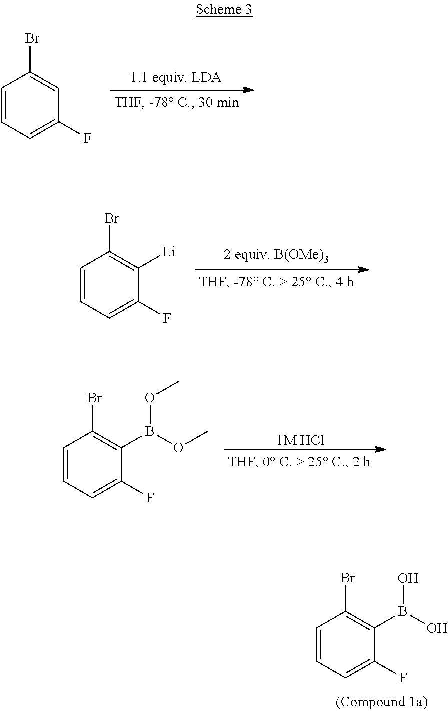

In a 500 mL three necked flask, with condenser, nitrogen purge inlet and an inserted digital thermometer, diisopropylamine (8.90 g, 13 mL, 1.1 equiv., 88.0 mmol) was dissolved in tetrahydrofuran (200 mL, abs, N₂ purged) and was cooled to −78° C. The solution of butyllithium (5.64 g, 35.2 mL, 1.1 equiv., 88.0 mmol, 2.5 M in hexanes) was added dropwise, keeping the reaction temperature below −60° C. The reaction mixture was stirred for 30 min at −78° C. Then, 1-bromo-3-fluorobenzene (14.0 g, 8.93 mL, 1 equiv., 80.0 mmol) was added dropwise within 5 min, keeping the reaction temperature under −70° C. The mixture was stirred for 30 min at −78° C. Then, trimethyl borate (16.6 g, 18 mL, 2 equiv., 160 mmol) was added dropwise within 10 min and the reaction temperature was maintained below −70° C. The reaction was then stirred for 30 min at −78° C., left to warm up to 25° C. and stirred for another 4 h. Afterwards, the reaction mixture was cooled down to 0° C. and 250 mL 1M HCl solution (precooled to 0° C.) was added dropwise, keeping the temperature below 6° C. The reaction was left to warm up to 25° C. and stirred for another 2 h. Then, 160 mL of diethyl ether was added, and the phases were separated.

The aqueous phase was washed with another 40 mL of diethyl ether. The combined organic phase was washed with 2×160 mL brine and dried using Na₂SO₄. Finally, the solvents were evaporated on a rotary evaporator yielding a crude crystalline product, which can be used for the next synthetic step without further purification.

Step b) Synthesis of potassium (2-bromo-6-fluorophenyl)trifluoroborate (Compound 1b)

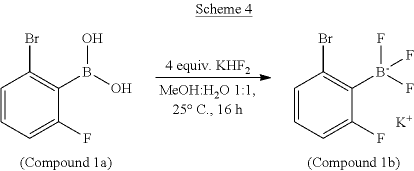 [SMILES: OB(O)c1c(F)cccc1Br ; "4 equiv. KHF2" ; Fc1cccc(Br)c1[B-](F)(F)F ; [K+]]

In a white 1000 mL polypropylene container, (2-bromo-6-fluorophenyl)boronic acid (Compound 1a) (17.5 g, 1 equiv., 80.0 mmol) was measured in and dissolved in methanol (90 mL, tech). Then, potassium hydrogen fluoride (25.0 g, 4 equiv., 320 mmol) dissolved in water (90 mL) was added in one portion. The resulting suspension was stirred for 16 h. Afterwards, 500 mL of acetone was added and the reaction mixture was stirred for 30 min. The reaction mixture was filtered through filter paper and the solvents were evaporated at 60° C. on a rotary evaporator. Additional 400 mL acetone was added and evaporated again to remove the traces of water. Finally, 100 mL toluene was added and evaporated the same way. The obtained white powder was dissolved once again in 100 mL acetone and filtered through filter paper. The filtrate was evaporated, and the obtained white powder was mixed with 100 mL of hexanes and filtered. The precipitate was washed with 2×50 mL diethyl ether, dried on a rotary evaporator at 60° C. and kept in a vacuum desiccator using P₄O₁₀ as desiccant. The product is a white, crystalline solid (20.4 g, 72.6 mmol). The combined isolated yield for the first two synthetic steps is 91%.

### ¹H NMR

¹H NMR (300 MHz, DMSO-d6) δ 7.18 (dq, J=7.8, 0.6 Hz, 1H), 6.99 (td, J=8.0, 6.1 Hz, 1H), 6.85 (dddt, J=9.4, 8.1, 1.2, 0.6 Hz, 1H).

### ¹⁹F NMR

¹⁹F NMR (282 MHz, DMSO-d6) δ −96.2 (tdd, J=16.1, 12.6, 6.8 Hz, 1F), −127.6-−128.6 (m, 3F).

### ¹³C NMR

Partial ¹³C NMR (75 MHz, Benzene-d6) δ 165.8 (d, J=244.8 Hz, 1C), 128.4 (d, J=3.2 Hz, 1C), 128.4 (d, J=9.6 Hz, 1C), 128.0 (d, J=14.0 Hz, 1C), 113.7 (d, J=27.9 Hz, 1C).

Step c) Synthesis of (2-bromo-6-fluorophenyl)bis(2,3,5,6-tetrafluorophenyl)borane (Compound 1)

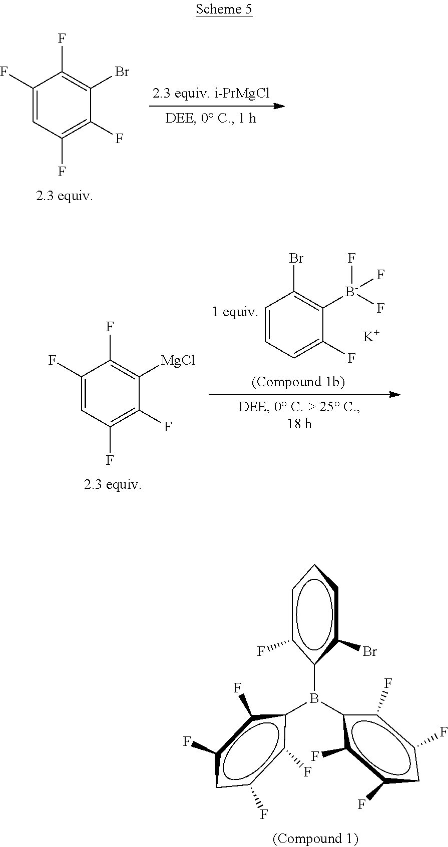

A 100 mL 3-necked flask was equipped with a reflux condenser and N₂ inlet, magnesium turnings (1.61 g, 2.3 equiv., 66.3 mmol) were measured in and activated with iodine. Then, 20 mL of abs. diethyl ether was added followed by the dropwise addition of 2-chloropropane (5.21 g, 6.04 mL, 2.3 equiv., 66.3 mmol). The solution started to warm up and reflux. 30 mL of diethyl ether was added to dilute the reaction, and dropwise addition of 2-chloropropane was continued to maintain the reflux. In another 250 mL 2-necked flask, 3-bromo-1,2,4,5-tetrafluorobenzene (15.2 g, 8.07 mL, 2.3 equiv., 66.3 mmol) was measured in and dissolved in 90 mL of abs. diethyl ether, after which it was cooled to 0° C. The previously prepared Grignard solution was added dropwise via syringe within 45 min, while keeping the reaction temperature below 5° C. After completion of the addition, the reaction mixture was stirred for 1 h. In a 500 mL Schlenk flask, potassium (2-bromo-6-fluorophenyl)trifluoroborate (Compound 1b) (8.10 g, 1 equiv., 28.8 mmol) was measured in under N₂, suspended in 20 mL of abs. diethyl ether and cooled down to 0° C. The cool (0° C.) Grignard solution was added via cannula within 20 min, while keeping the temperature under 4° C. The reaction mixture was left to warm up to 25° C. and was stirred for an additional 18 h. Afterwards, the solvent was evaporated at 50° C. in vacuo. Next, 90 mL of abs. toluene was added, and the suspension was sonicated for 10 minutes. The resulting precipitate was filtered off and washed with 2×20 mL of abs. toluene. The combined filtrate was then evaporated at 70° C. in vacuo, resulting an off-white solid. Then, 10 mL of abs. pentane was added, and the resulting suspension was sonicated and filtered to give the product as a white crystalline powder (6.90 g, 14.3 mmol, 50% yield).

### ¹H NMR

¹H NMR (300 MHz, Benzene-d6) δ 6.98-6.89 (m, 1H), 6.59-6.48 (m, 2H), 6.29-6.15 (m, 2H).

### ¹⁹F NMR

¹⁹F NMR (282 MHz, Benzene-d6) δ −102.1-−102.2 (m, 1F), −129.1-−129.3 (m, 4F), −138.0-−138.2 (m, 4F).

¹³C NMR

¹³C NMR (75 MHz, Benzene-d6) δ 162.8 (d, J=245.8 Hz, 1C), 148.4 (dddd, J=251.4, 12.8, 8.6, 3.7 Hz, 4C), 146.0 (dm, J=250.2 Hz, 4C), 133.2 (d, J=9.0 Hz, 1C), 132.4-131.5 (m, 1C), 128.3 (d, J=3.2 Hz, 1C), 123.4 (d, J=9.3 Hz, 1C), 120.4-118.8 (m, 2C), 114.1 (d, J=23.2 Hz, 1C), 111.8 (tt, J=22.7, 2.0 Hz, 2C).

### Example 2

### Synthesis of (2-bromo-6-fluorophenyl)bis(2,3,6-trifluorophenyl)borane (Compound 2, see Entry 16)

The compound was prepared as described below and illustrated in schemes 3, 4 and 6.

Step a) and Step b) are analogues to EXAMPLE 1.

Step c) Synthesis Of (2-bromo-6-fluorophenyl)bis(2,3,6-trifluorophenyl)borane (Compound 2)

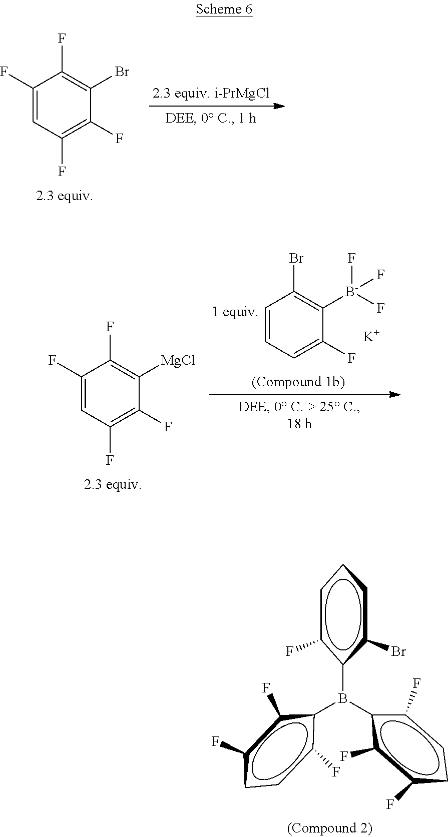

A 100 mL 3-necked flask was equipped with a reflux condenser and N₂ inlet, Magnesium turnings (1.61 g, 2.3 equiv., 66.3 mmol) were measured in and activated with iodine. Then, 20 mL abs. diethyl ether was added followed by the dropwise addition of 2-chloropropane (5.21 g, 6.04 mL, 2.3 equiv., 66.3 mmol). The solution started to warm up and reflux. Additional 30 mL diethyl ether was added, and dropwise addition of 2-chloropropane was continued to maintain the reflux. In another 250 mL 2-necked flask, 2-bromo-1,3,4-trifluorobenzene (14.0 g, 7.85 mL, 2.3 equiv., 66.3 mmol) was measured in and dissolved in 90 mL abs. diethyl ether, after which it was cooled to 0° C. The previously prepared Grignard solution was added dropwise via syringe in 45 min, keeping the reaction temperature below 5° C. After completion of the addition, the reaction mixture was stirred for 1 h. In a 500 mL Schlenk flask, potassium (2-bromo-6-fluorophenyl)trifluoroborate (Compound 1b) (8.10 g, 1 equiv., 28.8 mmol) was measured in under N₂, suspended in 20 mL abs. diethyl ether and cooled down to 0° C. The cool (0° C.) Grignard solution was added via cannula within 20 min, while keeping the temperature under 4° C. The reaction mixture was left to warm up to 25° C. and was stirred for an additional 18 h. Afterwards, the solvent was evaporated at 50° C. in vacuo. Next, 90 mL abs. toluene was added, and the suspension was sonicated for 10 minutes. The resulting precipitate was filtered off and washed with 2×20 mL abs. toluene. The combined filtrate was then evaporated at 70° C. in vacuo, resulting an off-white solid. Then, 10 mL abs. pentane was added, and the resulting suspension was filtered to yield the product as a white crystalline powder (8.05 g, 18.0 mmol, 63% yield)

### ¹H NMR

¹H NMR (300 MHz, Benzene-d6) δ 7.04-6.95 (m, 1H), 6.65-6.44 (m, 4H), 6.21-6.11 (m, 2H).

### ¹⁹F NMR

¹⁹F NMR (282 MHz, Benzene-d6) δ −102.4-−102.5 (m, 1F), −103.4-−103.6(m, 2F), −123.0 (ddt, J=21.9, 9.2, 1.7 Hz, 2F), −142.6 (dddd, J=21.9, 15.8, 9.4, 3.3 Hz, 2F).

### ¹³C NMR

¹³C NMR (75 MHz, Benzene-d6) δ 162.9 (d, J=245.1 Hz, 1C), 160.7 (ddd, J=250.1, 8.5, 2.5 Hz, 2C), 153.0 (ddd, J=254.6, 13.3, 11.4 Hz, 2C), 147.3 (ddd, J=246.0, 14.7, 3.5 Hz, 2C), 133.9-132.4 (m, 1C), 132.5 (d, J=8.9 Hz, 1C), 128.2 (d, J=3.1 Hz, 1C), 123.6 (d, J=9.7 Hz, 1C), 122.9 (ddd, J=19.6, 11.3, 2.5 Hz, 2C), 120.2-118.9 (m, 2C), 113.9 (d, J=23.3 Hz, 1C), 111.5 (ddd, J=27.5, 5.8, 4.1 Hz, 2C).

### Example 3

### Synthesis of (2-bromo-6-fluorophenyl)bis(perfluorophenyl)borane (Compound 3, See Entry 11).

The compound was prepared as described below and illustrated in schemes 3, 4 and 7.

Step a) and Step b) are analogues to EXAMPLE 1.

Step c) Synthesis of (2-bromo-6-fluorophenyl)bis(perfluorophenyl)borane (Compound 3)

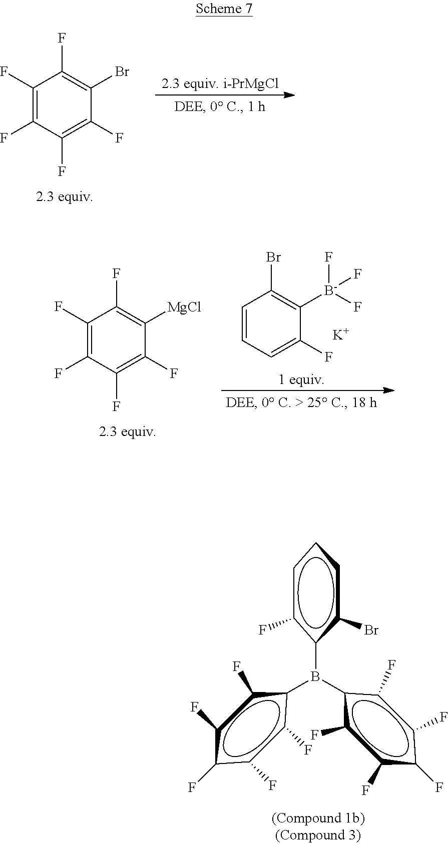

A 100 mL 3-necked flask was equipped with a reflux condenser and N₂ inlet, Magnesium turnings (1.61 g, 2.3 equiv., 66.3 mmol) were measured in and activated with iodine. Then, 20 mL abs. diethyl ether was added followed by the dropwise addition of 2-chloropropane (5.21 g, 6.04 mL, 2.3 equiv., 66.3 mmol). The solution started to warm up and reflux. Additional 30 mL diethyl ether was added, and dropwise addition of 2-chloropropane was continued to maintain the reflux. In another 250 mL 2-necked flask, 1-bromo-2,3,4,5,6-pentafluorobenzene (16.4 g, 8.27 mL, 2.3 equiv., 66.3 mmol) was measured in and dissolved in 90 mL abs. diethyl ether, after which it was cooled to 0° C. The previously prepared Grignard solution was added dropwise via syringe in 45 min, keeping the reaction temperature below 5° C. After completion of the addition, the reaction mixture was stirred for 1 h. In a 500 mL Schlenk flask, potassium (2-bromo-6-fluorophenyl)trifluoroborate (Compound 1b) (8.10 g, 1 equiv., 28.8 mmol) was measured in under N₂ suspended in 20 mL abs. diethyl ether and cooled down to 0° C. The cool (0° C.) Grignard solution was added via cannula within 20 min, while keeping the temperature under 4° C. The reaction mixture was left to warm up to 25° C. and stirred for an additional 18 h. Afterwards, the solvent was evaporated at 50° C. in vacuo. Next, 90 mL abs. toluene was added and the suspension was sonicated for 10 minutes. The resulting precipitate was filtered off and washed with 2×20 mL abs. toluene. The combined filtrate was then evaporated at 70° C. in vacuo, resulting an off-white solid. Then, 10 mL abs. pentane was added, and the resulting suspension was filtered to yield the product as a white crystalline powder (5.08 g, 9.79 mmol, 34% yield).

### ¹H NMR

¹H NMR (300 MHz, Benzene-d6) δ 6.99-6.91 (m, 1H), 6.64-6.55 (m, 2H).

### ¹⁹F NMR

¹⁹F NMR (282 MHz, Benzene-d6) δ −102.4-−102.5 (m, 1F), −127.9-−128.1 (m, 4F), −142.9 (tt, J=20.9, 7.0 Hz, 2F), −160.6-−160.87 (m, 4F).

### ¹³C NMR

¹³C NMR (75 MHz, Benzene-d6) δ 162.7 (d, J=245.4 Hz, 1C), 149.2 (dtt, J=252.6, 10.9, 4.2 Hz, 4C), 145.2 (dm, J=261.8 Hz, 2C), 137.7 (dm, J=256.1 Hz, 4C), 133.2 (d, J=9.1 Hz, 1C), 132.4-131.3 (m, 1C), 128.3 (d, J=3.1 Hz, 1C), 123.2 (d, J=9.4 Hz, 1C), 114.1 (d, J=23.1 Hz, 1C), 114.3-112.9 (m, 2C).

### Example 4

### Synthesis of (perfluoro-[1,1′-biphenyl]-2-yl)bis(2,4,6-trifluorophenyl)borane (Compound 4, See Entry 15)

The compound was prepared as described below and illustrated in schemes 8-10.

Step a) Synthesis of (perfluoro-[1,1′-biphenyl]-2-yl)boronic acid (Compound 4a, Pfp=C₆F₅)

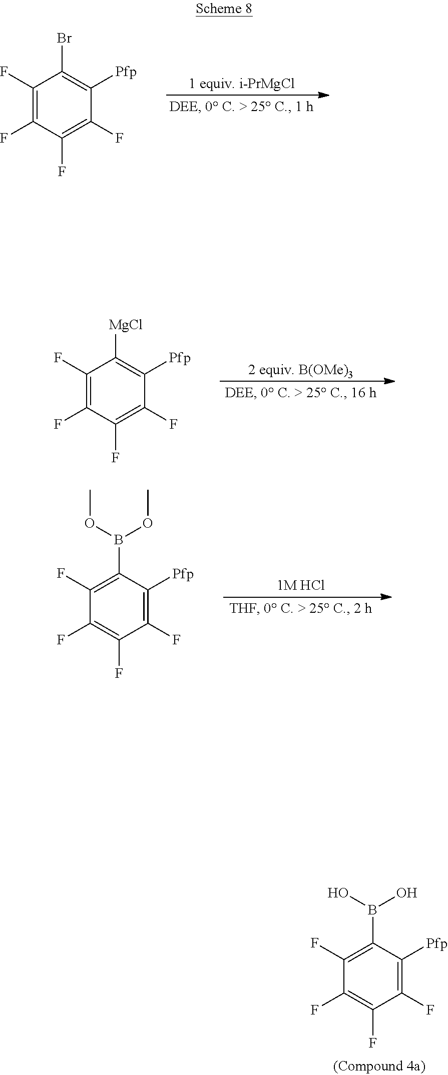

Preparation of i-PrMgCl: A 100 mL 3-necked flask was equipped with a reflux condenser and N₂ inlet, magnesium turnings (1.89 g, 1.0 equiv., 77.7 mmol) were measured in and 45 mL abs. diethyl ether was added. Then 2-chloropropane (6.11 g, 7.11 mL, 1.0 equiv., 77.7 mmol) was added dropwise. The solution started to warm up and reflux. The dropwise addition of 2-chloropropane was continued to maintain the reflux.

In a 500 mL three necked flask, with condenser, nitrogen purge inlet and an inserted digital thermometer, 2-bromo-2′,3,3′,4,4′,5,5′,6,6′-nonafluoro-1,1′-biphenyl (30.7 g, 1.0 equiv., 77.7 mmol) was dissolved in diethyl ether (50 mL, abs, N₂ purged) and cooled to 0° C. with an ice bath. The solution of i-PrMgCl was added dropwise, keeping the reaction temperature between 0 and 5° C. Then the reaction mixture was stirred for 60 minutes at 25° C. Then, trimethyl borate (16.2 g, 17.7 mL, 2.0 equiv., 155 mmol) was added dropwise within 30 min, keeping the reaction temperature at 0° C. The mixture was stirred for an additional 16 hours at 25° C. Afterwards, the reaction was cooled down to 0° C. and 80 mL 1M HCl solution (precooled to 0° C.) was added dropwise, keeping the temperature below 5° C. The reaction was left to warm up to 25° C. and stirred for another 2 h. Then, 200 mL diethyl ether was added, and the phases were separated. The aqueous phase was washed with another 50 mL diethyl ether. The combined organic phase was washed with 2×160 mL brine and dried using Na₂SO₄. Finally, the solvents were evaporated on a rotary evaporator yielding a crude product, which can be used for the next synthetic step without further purification.

Step b) Synthesis of potassium trifluoro(perfluoro-[1,1′-biphenyl]-2-yl)borate (Compound 4b)

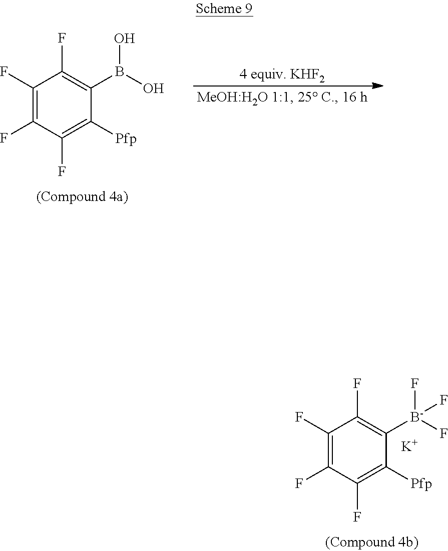

In a white 1000 mL polypropylene container, (perfluoro-[1,1′-biphenyl]-2-yl)boronic acid (26.59 g, 77.7 mmol) was measured in and dissolved in methanol (78 mL, tech). Then, potassium hydrogen fluoride (24.30 g, 4,0 equiv., 311.11 mmol) dissolved in distilled water (78 mL) was added in one portion. The resulting suspension was stirred for 16 h. Afterwards, 500 mL acetone was added. The reaction mixture was filtered through filter paper, and the solvents were evaporated at 60° C. on a rotary evaporator. Additional 400 mL acetone was added and evaporated again to remove the traces of water. Then, 100 mL toluene was added and evaporated the same way. The obtained white powder was dissolved once again in 100 mL acetone and filtered through filter paper. The solvent was evaporated on a rotary evaporator, and the obtained white powder was mixed with 100 mL of hexanes, filtered, then dried at 60° C. The product is a white, crystalline solid (25.73 g, 63.68 mmol). The isolated yield for this synthetic step is 81.9%. equiv.

### ¹⁹F NMR

¹⁹F NMR (282 MHz, DMSO-d6) δ −132.5-−132.8 (m, 1F), −134.0-−134.74 (m, 3F), −139.9-−140.1 (m, 2F), −141.0 (dd, J=22.5, 13.8 Hz, 1F), −155.8 (t, J=22.1 Hz, 1F), −156.0 (dd, J=25.2, 20.9 Hz, 1F), −160.9 (ddd, J=22.9, 20.9, 2.7 Hz, 1F), −164.4-−164.68 (m, 2F).

### ¹³C NMR

Partial ¹³C NMR (75 MHz, DMSO-d6) δ 148.7 (d, J=240.7 Hz, 1C), 144.2 (dm, J=244.8 Hz, 2C), 143.7 (dm, J=243.2 Hz, 1C), 140.0 (dm, J=255.6 Hz, 1C), 137.6 (dm, J=245.9 Hz, 1C), 136.4 (dm, J=249.1 Hz, 2C).

Step c) Synthesis of (2-bromo-6-fluorophenyl)bis(2,4,6-trifluorophenyl)borane (Compound 4)

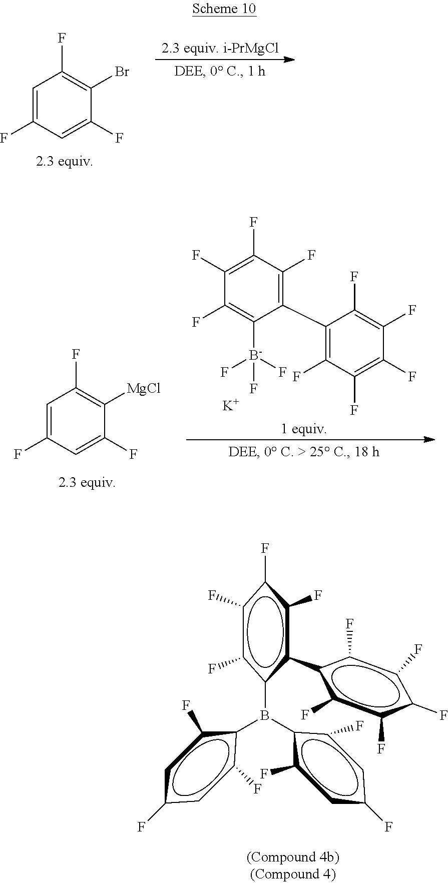

A 100 mL 3-necked flask was equipped with a reflux condenser and N₂ inlet, magnesium turnings (280 mg, 2.3 equiv., 11.5 mmol) were measured in. Then, 10 mL abs. diethyl ether was added followed by the dropwise addition of 2-chloropropane (903 mg, 1.05 mL, 2.3 equiv., 11.5 mmol). The solution started to warm up and reflux. Additional 10 mL diethyl ether was added, and dropwise addition of 2-chloropropane was continued to maintain the reflux. In another 100 mL 2-necked flask, 2-bromo-1,3,5-trifluorobenzene (2.43 g, 1.355 mL, 2.3 equiv., 11.5 mmol) was measured in and dissolved in 40 mL abs. diethyl ether, after which the solution was cooled to 0° C. The previously prepared i-PrMgCl solution was added dropwise via syringe within 20 min, keeping the reaction temperature below 5° C. After completion of the addition, the reaction mixture was stirred for 1 h. In a 100 mL Schlenk flask, potassium trifluoro(perfluoro-[1,1′-biphenyl]-2-yl)borate (Compound 4b) (2.11 g, 1 equiv., 5.00 mmol) was measured in under N₂, suspended in 5 mL abs. diethyl ether and cooled down to 0° C. The cool (0° C.) Grignard solution was added via cannula within 20 min, while keeping the temperature under 5° C. The reaction mixture was left to warm up to 25° C. and was stirred for an additional 18 h. Afterwards, the solvent was evaporated in vacuo. Next, 10 mL abs. toluene was added, and the suspension was sonicated for 10 minutes. The resulting precipitate was filtered off and washed with 2×5 mL abs. toluene. The combined filtrate was then evaporated at 45° C. in vacuo, resulting an off-white solid. Then, 10 mL abs. pentane was added, and the resulting suspension was filtered to yield the product as an off white crystalline powder (1.33 g, 2.27 mmol, 45% yield).

### ¹H NMR

¹H NMR (300 MHz, Benzene-d6) δ 6.02-5.91 (m, 4H).

### ¹⁹F NMR

¹⁹F NMR (282 MHz, Benzene-d6) δ −94.2 (ddt, J=12.4, 8.4, 4.1 Hz, 4F), −97.0-−97.2 (m, 2F), −129.0 (ddd, J=22.8, 12.2, 6.6 Hz, 1F), −136.5-−136.8 (m, 1F), −139.4-−139.7 (m, 2F), −148.63 (td, J=20.7, 6.7 Hz, 1F), −151.8-−152.0 (m, 1F), −152.9 (ddd, J=22.9, 20.2, 5.8 Hz, 1F), −161.9-−162.2 (m, 2F).

### ¹³C NMR

Partial ¹³C NMR (75 MHz, Benzene-d6) δ 167.6 (dt,J=258.3, 16.9 Hz, 2C), 166.6 (ddd, J=254.7, 15.3, 13.7 Hz, 4C), 149.1 (dm, J=246.4 Hz, 1C), 146.4 (ddd, J=251.2, 10.9, 3.5 Hz, 1C), 144.4 (dddt, J=249.2, 10.6, 7.0, 4.0 Hz, 2C), 142.7 (dddd, J=259.3, 17.3, 12.5, 4.9 Hz, 1C), 141.7 (dm, J=256.6 Hz, 2C), 137.7 (dm, J=254.7 Hz, 2C), 114.5-113.4 (m, 2C), 113.2-112.7 (m, 1C), 108.9-108.2 (m, 1C), 100.6 (ddd, J=28.9, 25.0, 3.6 Hz, 4C).

### Example 5

### Synthesis of (2-bromo-6-fluorophenyl)bis(2,4,6-trifluorophenyl)borane (Compound 5, See Entry 19)

The compound was prepared as described below and illustrated in schemes 3, 4 and 11.

Step a) and Step b) are analogues to EXAMPLE 1.

Step c) Synthesis of (2-bromo-6-fluorophenyl)bis(2,4,6-trifluorophenyl)borane (Compound 5)

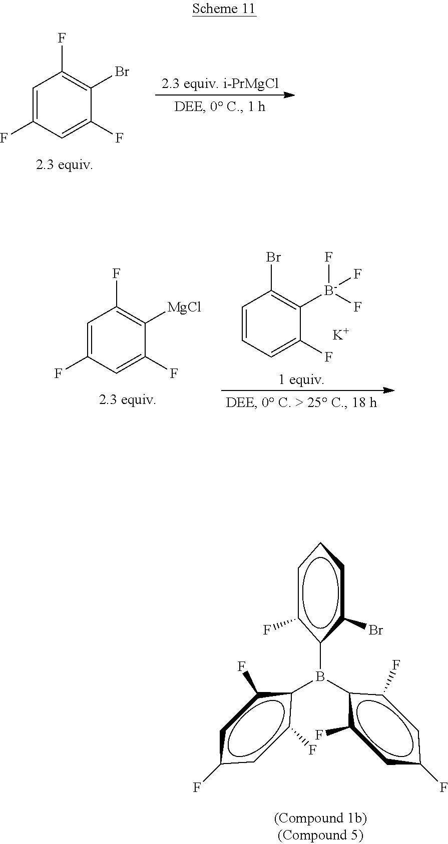

A 100 mL 3-necked flask was equipped with a reflux condenser and N₂ inlet, Magnesium turnings (1.61 g, 2.3 equiv., 66.3 mmol) were measured in and activated with iodine. Then, 20 mL abs. diethyl ether was added followed by the dropwise addition of 2-chloropropane (5.21 g, 6.04 mL, 2.3 equiv., 66.3 mmol). The solution started to warm up and reflux. Additional 30 mL diethyl ether was added, and dropwise addition of 2-chloropropane was continued to maintain the reflux. In another 250 mL 2-necked flask, 2-bromo-1,3,5-trifluorobenzene (14.0 g, 7.82 mL, 2.3 equiv., 66.3 mmol) was measured in and dissolved in 90 mL abs. diethyl ether, after which it was cooled to 0° C. The previously prepared Grignard solution was added dropwise via syringe in 45 min, keeping the reaction temperature below 5° C. After completion of the addition, the reaction mixture was stirred for 1 h. In a 500 mL Schlenk flask, potassium (2-bromo-6-fluorophenyl)trifluoroborate (Compound 1b) (8.10 g, 1 equiv., 28.8 mmol) was measured in under N₂ suspended in 20 mL abs. diethyl ether and cooled down to 0° C. The cool (0° C.) Grignard solution was added via cannula within 20 min, while keeping the temperature under 4° C. The reaction mixture was left to warm up to 25° C. and was stirred for an additional 18 h. Afterwards, the solvent was evaporated at 50° C. in vacuo. Next, 90 mL abs. toluene was added, and the suspension was sonicated for 10 minutes. with the resulting precipitate was filtered off and washed with 2×20 mL abs. toluene. The combined filtrate was then evaporated at 70° C. in vacuo, resulting an off-white solid. Then, 10 mL abs. pentane was added, and the resulting suspension was filtered to yield the product as a white crystalline powder (6.05 g, 13.5 mmol, 47% yield).

### ¹H NMR

¹H NMR (500 MHz, Benzene-d6) δ 7.04 (dd, J=7.5, 1.3 Hz, 1H), 6.68-6.60 (m, 2H), 6.14-6.07 (m, 4H).

### ¹⁹F NMR

¹⁹F NMR (282 MHz, Benzene-d6) δ −94.4 (td, J=9.8, 2.1 Hz, 4F), −98.7 (tt, J=11.5, 8.8 Hz, 2F), −102.8-−102.9 (m, 1F).

### ¹³C NMR

¹³C NMR (126 MHz, Benzene-d6) δ 167.4 (dt, J=256.6, 16.9 Hz, 2C), 167.1 (ddd, J=254.8, 15.4, 13.6 Hz, 4C), 162.8 (d, J=244.4 Hz, 1C), 134.2-133.5 (m, 1C), 132.0 (d, J=8.7 Hz, 1C), 128.1 (d, J=3.0 Hz, 1C), 123.7 (d, J=10.0 Hz, 1C), 114.9-114.1 (m, 2C), 113.8 (d, J=23.5 Hz, 1C), 100.6 (ddd, J=30.1, 24.9, 3.2 Hz, 4C)

### Example 6

### Synthesis of (2-chloro-6-fluorophenyl)bis(2,3,5,6-tetrafluorophenyl)borane (Compound 6, See Entry 10)

The compound was prepared as described below and illustrated in schemes 12 to 14.

Step a) Synthesis of (2-chloro-6-fluorophenyl)boronic acid (Compound 6a)

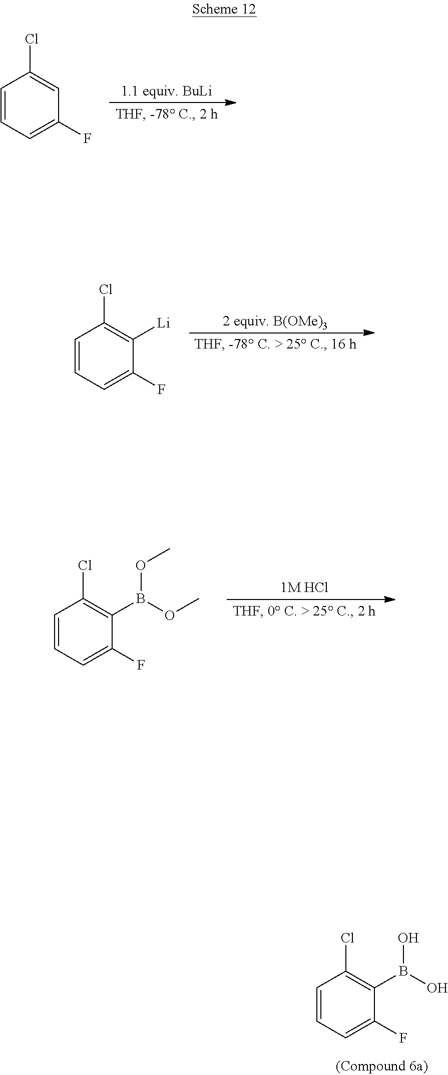

In a 500 mL necked flask, with condenser, nitrogen purge inlet and an inserted digital thermometer, at −75° C., 1-chloro-3-fluorobenzene (15.7 mL, 19.142 g, 146.6 mmol) was added to a solution of butyllithium (161.3 mmol) in tetrahydrofuran (117 mL, abs) and hexanes (66 mL, abs). The reaction mixture was stirred for 2 hours at −78° C. Then, trimethyl borate (30.47 g, 32.7 mL, 2 equiv., 40.0 mmol) was added dropwise within 50 min and the reaction temperature was maintained below −71° C. The reaction was left to warm up to 25° C. and then stirred for 16 hours. Afterwards, the reaction was cooled down to 0° C. and 35 mL 1M HCl solution (precooled to 0° C.) was added dropwise, keeping the temperature below 6° C. The reaction was left to warm up to 25° C. and stirred for another 2 h. Then, the phases were separated. The aqueous phase was washed with another 30 mL diethyl ether. The combined organic phase was washed with 2×30 mL brine and dried using Na₂SO₄. Finally, the solvents were evaporated on a rotary evaporator yielding a nearly solid, which was washed with hexane and dried. The product was obtained as a white powder (20.76 g, 119.06 mmol). The yield of this synthetic step is 81%. The crude product can be used for the next synthetic step without further purification.

Step b) Synthesis of potassium (2-chloro-6-fluorophenyl)trifluoroborate (Compound 6b)

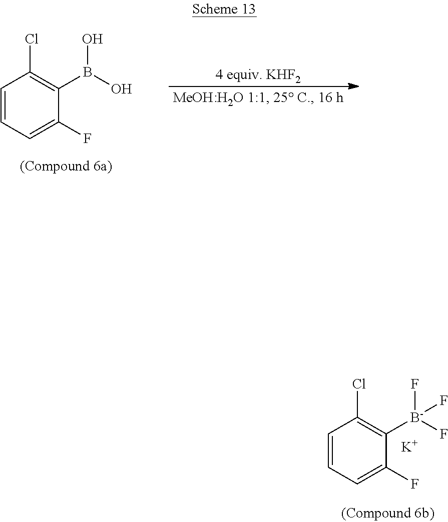 [SMILES: OB(O)c1c(F)cccc1Cl ; "4 equiv. KHF2" ; "MeOH:H2O 1:1, 25° C., 16 h" ; Fc1cccc(Cl)c1[B-](F)(F)F ; [K+]]

In a white 1000 mL polypropylene container, (2-chloro-6-fluorophenyl)boronic acid (Compound 6a) (20.76 g, 1 equiv., 119.06 mmol) was measured in and dissolved in methanol (325 mL, tech). Then, potassium hydrogen fluoride (37.2 g, 4 equiv., 476.22 mmol) dissolved in water (325 mL) was added in one portion. The resulting suspension was stirred for 16 h.

Afterwards, 300 mL acetone was added, and the reaction mixture was stirred for 30 min. The reaction mixture was filtered through filter paper and the solvents were evaporated at 60° C. on a rotary evaporator. Additional 2×30 mL acetone was added and evaporated again to remove the traces of water. Finally, 50 mL toluene was added and evaporated the same way. The obtained white powder was dissolved once again in 100 mL acetone and filtered through filter paper. The solvent was evaporated on a rotary evaporator, and the obtained white powder was mixed with 100 mL hexanes and filtered. The filtride was dried on a rotary evaporator at 60° C. and kept in a vacuum desiccator using P₄O₁₀ as desiccant. The product is a white, crystalline solid (27.00 g, 114.19 mmol). The isolated yield for this synthetic step is 96%.

### ¹H NMR

¹H NMR (500 MHz, DMSO-d6) δ 7.09 (td, J=8.0, 6.2 Hz, 1H), 7.00 (d, J=7.8 Hz, 1H), 6.82 (t, J=8.7 Hz, 1H).

### ¹⁹F NMR

¹⁹F NMR (282 MHz, DMSO-d6) δ −102.2- −102.5 (m, 1F), −132.2-−132.9 (m, 3F).

### ¹³C NMR

¹³C NMR (126 MHz, DMSO-d6) δ 166.0 (d, J=243.0 Hz, 1C), 138.9 (d, J=14.8 Hz, 1C), 134.2-131.3 (m, 1C), 128.0 (d, J=10.0 Hz, 1C), 125.0 (d, J=3.4 Hz, 1C), 113.2 (d, J=27.9 Hz, 1C).

Step c) Synthesis of (2-chloro-6-fluorophenyl)bis(2,3,5,6-tetrafluorophenyl)borane (Compound 6)

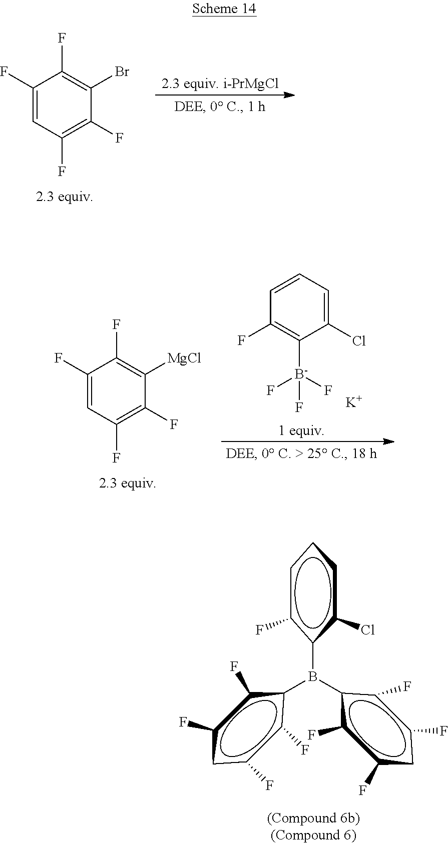

A 100 mL 3-necked flask was equipped with a reflux condenser and N₂ inlet, Magnesium turnings (763 mg, 2.3 equiv., 31.4 mmol) were measured in and activated with iodine. Then, 18 mL abs. diethyl ether was added followed by the dropwise addition of 2-chloropropane (2.47 g, 2.90 mL, 2.3 equiv., 31.4 mmol). The solution started to warm up and reflux. Additional 30 mL diethyl ether was added, and dropwise addition of 2-chloropropane was continued to maintain the reflux. In another 250 mL 2-necked flask, 3-bromo-1,2,4,5-tetrafluorobenzene (7.19 g, 2.3 equiv., 31.4 mmol) was measured in and dissolved in 95 mL abs. diethyl ether, after which the solution was cooled to 0° C. The previously prepared Grignard solution was added dropwise via syringe within 25 min, keeping the reaction temperature below 5° C. After completion of the addition, the reaction mixture was stirred for 1 h. In a 250 mL Schlenk flask, potassium (2-chloro-6-fluorophenyl)trifluoroborate (Compound 6b) (3.84 g, 1 equiv., 13.65 mmol) was measured in under N₂ suspended in 12 mL abs. diethyl ether and cooled down to 0° C. The cool (0° C.) Grignard solution was added via cannula within 20 min, while keeping the temperature under 4° C. The reaction mixture was left to warm up to 25° C. and was stirred for an additional 18 h. Afterwards, the solvent was evaporated at 50° C. in vacuo. Next, 60 mL abs. toluene was added, and the suspension was sonicated for 10 minutes. with the resulting precipitate was filtered off and washed with 2×10 mL abs. toluene. The combined filtrate was then evaporated at 70° C. invacuo, resulting an off-white solid. Then, 2×5 mL abs. pentane was added, and the resulting suspension was filtered to yield the product as a white crystalline powder (2.15 g, 4.89 mmol, 36% yield).

### ¹H NMR

¹H NMR (300 MHz, Benzene-d6) δ 6.77 (dt, J=8.0, 0.9 Hz, 1H), 6.62 (tdd, J=8.1, 6.4, 0.7 Hz, 1H), 6.51 (tt, J=8.4, 0.9 Hz, 1H), 6.25 (ttd, J=9.3, 7.5, 0.7 Hz, 2H).

### ¹⁹F NMR

¹⁹F NMR (282 MHz, Benzene-d6) δ −101.8-−101.9 (m, 1F), −129.5-−129.7 (m, 4F), −138.0-−138.2 (m, 4F).

### ¹³C NMR

¹³C NMR (75 MHz, Benzene-d6) δ 163.3 (d, J=246.1 Hz, 1C), 148.2 (dddd, J=250.8, 12.7, 8.7, 3.6 Hz, 4C), 146.1 (dm, J=250.3 Hz, 4C), 136.1 (d, J=9.9 Hz, 1C), 133.5 (d, J=9.6 Hz, 1C), 130.2 -128.8 (m, 1C), 125.5 (d, J=3.1 Hz, 1C), 120.6-119.2 (m, 2C), 113.8 (d, J=23.4 Hz. 1C), 111.6 (tt, J=22.7, 2.0 Hz, 2C).

### Example 7

### Synthesis of (perfluoro-[1,1′-biphenyl]-2-yl)bis(2,3,5,6-Tetrafluorophenyl)borane (Compound 7, See Entry 18)

The compound was prepared as described below and illustrated in schemes 8, 9 and 15.

Step a) and Step b) are analogues to EXAMPLE 4.

Step c) Synthesis of (perfluoro-[1,1′-Biphenyl]-2-yl)bis(2,3,5,6-tetrafluorophenyl)borane (Compound 7)

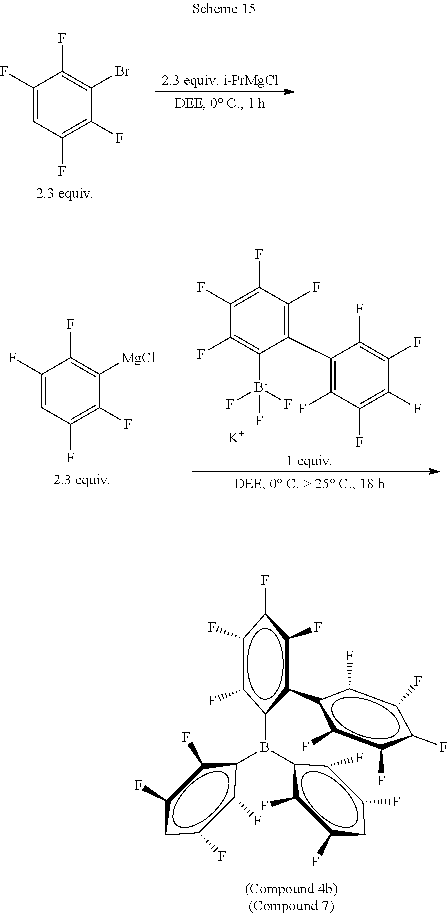

A 100 mL 3-necked flask was equipped with a reflux condenser and N₂ inlet, magnesium turnings (671 mg, 2.3 equiv., 27.6 mmol) were measured in and activated with iodine. Then, 20 mL abs. diethyl ether was added followed by the dropwise addition of 2-chloropropane (2.17 g, 2.52 mL, 2.3 equiv., 27.6 mmol). The solution started to warm up and reflux. Additional 30 mL diethyl ether was added, and dropwise addition of 2-chloropropane was continued to maintain the reflux. In another 250 mL 2-necked flask, 3-bromo-1,2,4,5-tetrafluorobenzene (6.32 g, 3.36 mL, 2.3 equiv., 27.6 mmol) was measured in and dissolved in 40 mL abs. diethyl ether, after which it was cooled to 0° C. The previously prepared i-PrMgCl solution was added dropwise via syringe within 25 min, keeping the reaction temperature below 5° C. After completion of the addition, the reaction mixture was stirred for 1 h. In a 250 mL Schlenk flask, potassium trifluoro(perfluoro-[1,1′-biphenyl]-2-yl)borate (Compound 4b) (5.10 g, 1 equiv., 12.0 mmol) was measured in under N₂ suspended in 15 mL abs. diethyl ether and cooled down to 0° C. The cool (0° C.) Grignard solution was added via cannula within 20 min, while keeping the temperature under 5° C. The reaction mixture was left to warm up to 25° C. and was stirred for an additional 18 h. Afterwards, the solvent was evaporated in vacuo. Next, 20 mL abs. toluene was added, and the suspension was sonicated for 10 minutes. The resulting precipitate was filtered off and washed with 2×15 mL abs. toluene. The combined filtrate was then evaporated at 45° C. in vacuo, resulting an off-white solid. Then, 20 mL abs. pentane was added, and the suspension was sonicated for 25 minutes. The resulting suspension was filtered to yield the product as an off-white crystalline powder (3.26 g, 5.22 mmol, 44% yield).

### ¹H NMR

¹H NMR (300 MHz, Benzene-d6) δ 6.15 (tt,J=9.3, 7.6 Hz, 2H).

### ¹⁹F NMR

¹⁹F NMR (282 MHz, Benzene-d6) δ −127.7 (ddd, J=22.9, 11.6, 7.5 Hz, 1F), −129.6 (bs, 4F), −135.4 (ddq, J=23.3, 11.7, 5.8 Hz, 1F), −137.7-−138.0 (m, 4F), −139.1-−139.4 (m, 2F), −146.4 (bs, 1F), −151.1 (t, J=21.0 Hz, 1F), −151.9 (td, J=21.5, 5.9 Hz, 1F), −161.3-−161.5 (m, 2F).

### ¹³C NMR

¹³C NMR (126 MHz, Benzene-d6)δ 149.9 (dd, J=246.8, 9.8 Hz, 1C), 147.7 (dddd, J=249.5, 12.9, 8.7, 3.6 Hz, 4C), 146.5 (dd, J=251.3, 11.3 Hz, 1C), 146.0 (dddd, J=251.6, 16.3, 8.9, 3.6 Hz, 4C), 144.5 (dm, J=248.4 Hz, 2C), 143.2 (dm, J=261.0 Hz, 1C), 142.0 (dtt, J=258.0, 13.2, 4.8 Hz, 1C), 141.5 (dddd, J=260.1, 19.6, 12.1, 3.3 Hz, 1C), 137.7 (dddd, J=253.1, 16.4, 12.4, 4.0 Hz, 2C), 126.5 (d, J=20.1 Hz, 1C), 119.7 (t, J=21.5 Hz, 2C), 113.7-113.4 (m, 1C), 111.1 (t, J=10 22.6 Hz, 2C), 108.1 (t, J=18.5 Hz, 1C).

### Example 8

### Synthesis of (perfluoro-[1,1′-biphenyl]-2-yl)bis(2,3,6-trifluorophenyl)borane (Compound 8, See Entry 17)

The compound was prepared as described below and illustrated in schemes 8, 9 and 16.

Step a) and Step b) are analogues to EXAMPLE 4.

Step c) Synthesis of (perfluoro-[1,1′-biphenyl]-2-yl)bis(2,3,6-trifluorophenyl)borane (Compound 8)

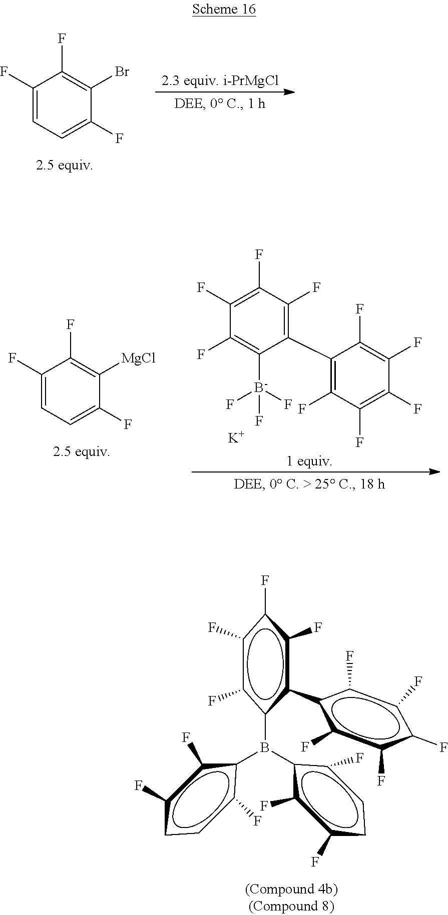

A 100 mL 3-necked flask was equipped with a reflux condenser and N2 inlet, magnesium turnings (648 mg, 2.5 equiv., 25.66 mmol) were measured in and activated with iodine. Then, 25 mL abs. diethyl ether was added followed by the dropwise addition of 2-chloropropane (2.09 g, 2.43 mL, 2.5 equiv., 25.66 mmol). The solution started to warm up and reflux. Additional 30 mL diethyl ether was added, and dropwise addition of 2-chloropropane was continued to maintain the reflux. In another 250 mL 2-necked flask, 2-bromo-1,3,4-trifluorobenzene (5.62 g, 3.15 mL, 2.5 equiv., 25.66 mmol) was measured in and dissolved in 40 mL abs. diethyl ether, after which it was cooled to 0° C. The previously prepared i-PrMgCl solution was added dropwise via syringe within 25 min, keeping the reaction temperature below 5° C. After completion of the addition, the reaction mixture was stirred for 1 h. In a 250 mL Schlenk flask, potassium trifluoro(perfluoro-[1,1′-biphenyl]-2-yl)borate (4.50 g, 1 equiv., 10.66 mmol) was measured in under N₂, suspended in 10 mL abs. diethyl ether and cooled down to 0° C. The cool (0° C.) Grignard solution was added via cannula within 20 min, while keeping the temperature under 5 ° C. The reaction mixture was left to warm up to 25° C. and was stirred for an additional 18 h. Afterwards, the solvent was evaporated in vacuo. Next, 20 mL abs. toluene was added, and the suspension was sonicated for 10 minutes. The resulting precipitate was filtered off and washed with 2×15 mL abs. toluene. The combined filtrate was then evaporated at 45° C. in vacuo, resulting an off-white solid. Then, 20 mL abs. pentane was added, and the suspension was sonicated for 25 minutes. The resulting suspension was filtered to yield the product as a white crystalline powder (1.883 g, 3.202 mmol, 30% yield).

### ¹H NMR

¹H NMR (500 MHz, Benzene-d6) δ 6.36 (qd, J=9.2, 5.2 Hz, 2H), 5.99 (tdd, J=8.9, 3.3, 1.8 Hz, 2H).

### ¹⁹F NMR

¹⁹F NMR (282 MHz, Benzene-d6) δ -103.3 (2F), −122.59 (d, J=21.7 Hz, 2F), −127.77 (ddd, J=22.8, 12.1, 7.4 Hz, 1F), −135.96 (ddd, J=22.2, 12.0, 6.0 Hz, 1F), −139.4 (d, J=22.5 Hz, 2F), −142.1 (dddd, J=21.6, 15.7, 9.4, 3.3 Hz, 2F), −146.6 (d, J=22.8 Hz, 1F), −151.56 (t, J=21.3 Hz, 1F), −152.0 (ddd, J=22.9, 20.5, 5.9 Hz, 1F), −161.5-−161.72 (m, 2F).

### ¹³ C NMR

Partial ¹³C NMR (126 MHz, Benzene-d6,) δ 160.0 (ddd, J=249.9, 8.2, 2.6 Hz, 2C), 152.3 (ddd, J=254.5, 13.6, 11.1 Hz, 2C), 149.5 (dd, J=247.4, 9.7 Hz, 1C), 147.1 (ddd, J=247.4, 14.6, 3.6 Hz, 2C), 146.4 (dd, J=252.1, 11.7 Hz, 1C), 144.5 (dm, J=249.3 Hz, 2C), 143.3 (dm, J=260.7 Hz, 1C), 141.8 (dm, J=258.2 Hz, 1C), 141.5 (dm, J=259.3 Hz, 1C), 137.6 (dm, J=255.1 Hz, 2C), 123.2 (dd, J=19.4, 11.2 Hz, 2C), 119.4-118.6 (m, 2C), 113.6-113.0 (m, 1C), 111.5 (ddd, J=27.4, 5.9, 3.9 Hz, 2C), 108.3 (t, J=15.9 Hz, 1C).

### Example 9

### Reduction of Tung Oil

The major fatty acid component of tung oil is alpha-eleostearic acid (82%) containing 1 cis and 2 trans double bonds, all in conjugation. The isomerization and overreduction of these double bonds can be avoided be reducing the triglyceride directly through hydrosilylation using the BrF(F₃ₐ)₂ borane (Compound 2) as catalyst.

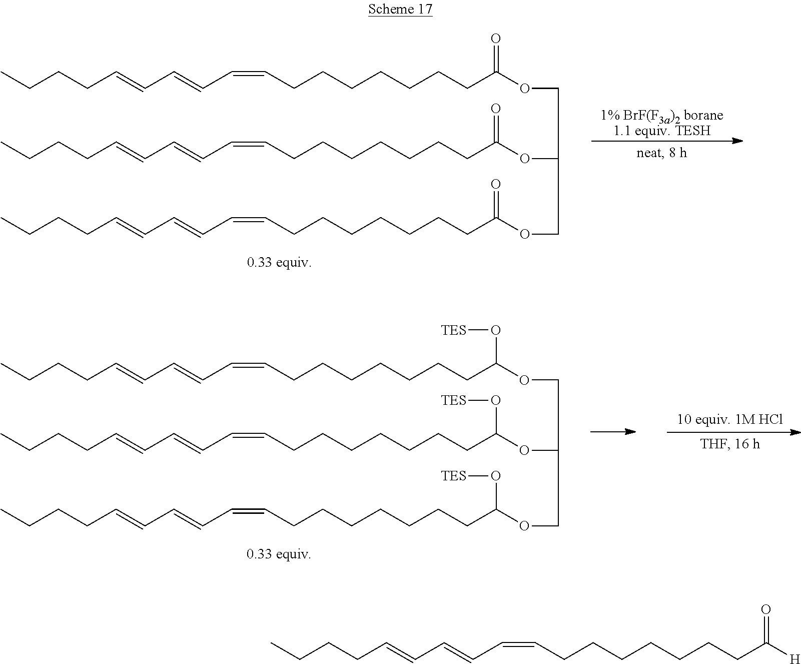

In an oven dried 4 mL vial the ester, propane-1,2,3-triyl (9Z,9′Z,9″Z,11E,11′E,11″E,13E,13′E,13″E)-tris(octadeca-9,11,13-trienoate) (146 mg, 0.33 equiv., 0.166 mmol) was measured in under nitrogen. Next, the solution of the catalyst in benzene-d6[(2-bromo-6-fluorophenyl)bis(2,3,6-trifluorophenyl)borane (Compound 2) (2.26 mg, 100 μL, 0.05 M in benzene-d6, 0.01 equiv., 5.0 μmol)] was added at room temperature. Then, under stirring, triethylsilane (64.4 mg, 89 μL, 1.1 equiv., 0.55 mmol) was added dropwise to the reaction mixture. The reaction was stirred overnight. The reaction went to complete conversion, as judged by ¹H NMR through the complete disappearance of the original glyceridic CH₂ peaks ((Benzene-d6) δ 4.29 (dd, J=11.9, 4.1 Hz, 2H), 4.07 (dd, J=11.9, 6.0 Hz, 2H)) and the appearance of the acetalic CH peak ((Benzene-d6) δ 5.03-4.93 (m, 2H)).

3,3,13,13-Tetraethyl-5,11-di((8Z,10E,12E)-heptadeca-8,10,12-trien-1-yl) -8-(((9Z,11E,13E)-1-((triethylsilyl)oxy)octadeca-9,11,13-trien-1-yl)oxy)-4,6,10,12-tetraoxa-3,13-disilapentadecane:

¹H NMR (500 MHz, Benzene-d6) δ 6.56 (dd, J=14.6, 11.3 Hz, 3H), 6.29-6.11 (m, 9H), 5.63 (dt, J=14.5, 7.1 Hz, 3H), 5.52-5.43 (m, 3H), 5.39-5.28 (m, 1H), 5.03-4.93 (m, 2H), 4.26-3.63 (m, 5H), 2.26-1.99 (m, 10H), 1.94-1.51 (m, 12H), 1.46-1.21 (m, 38H), 1.17-0.68 (m, 54H).

Next, the silyl acetal was hydrolysed by diluting the reaction mixture with 5 mL of THF and adding aqueous hydrochloric acid (182 mg, 5.0 mL, 1 M, 10 equiv., 5.0 mmol) to it. After 16 hours, the reaction mixture was extracted with 30 mL ethyl-acetate, washed with 30 mL saturated NaHCO₃ solution, dried over MgSO 4 and the solvent was removed under reduced pressure. The resulting oil contained alpha-eleostearaldehyde as a major constituent (>65 m/m %) and also minor contaminants from glycerol (<5 m/m %), hexaethyldisiloxane (<25 m/m %) and the other fatty acid components of tung oil (<5 m/m %) based on 1 HNMR. The aldehydic proton is clearly visible at δ9.33 (t, J=1.7 Hz, 1H), while the peaks of the silyl-acetal disappeared completely, indicating total conversion. The double bonds remained intact throughout the process as indicated by the olefinic H peaks ((Benzene-d6) δ 6.53 (dd, J=14.7, 11.2 Hz, 1H), 6.30-6.09 (m, 3H), 5.61 (dt, J=14.5, 7.1 Hz, 1H), 5.40 (dt, J=10.9, 7.7 Hz, 1H)).

(9Z,11E,13E)-Octadeca-9,11,13-trienal:

¹H NMR (500 MHz, Benzene-d6) δ 9.33 (t, J=1.8 Hz, 1H), 6.53 (dd, J=14.7, 11.2 Hz, 1H), 6.30-6.09 (m, 3H), 5.61 (dt,J=14.5, 7.1 Hz, 1H), 5.40 (dt, J=10.9, 7.7 Hz, 1H), 2.15 (qd, J=7.5, 1.5 Hz, 2H), 2.04-1.99 (m, 2H), 1.88-1.82 (m, 2H), 1.36-0.95 (m, 14H), 0.85 (t, J=7.1 Hz, 3H).

### Example 10

### Reduction of Jojoba Oil

Jojoba oil is composed almost entirely of mono-esters (wax esters). Its major fatty acid component is 11-eicosenoic acid, containing 1 double bond, and the major alcoholic components is 11-eicosanol.

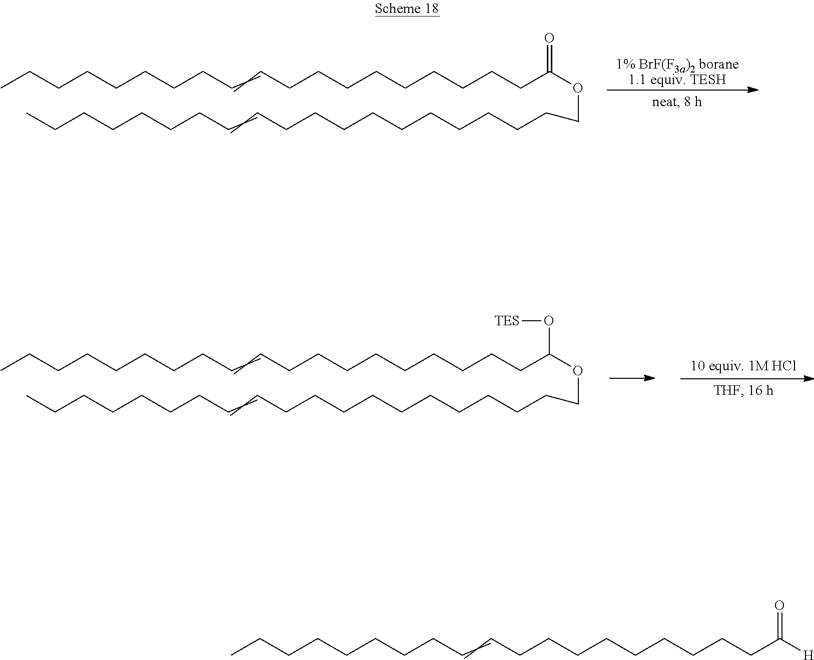

In an oven dried 4 mL vial the ester, icos-11-en-1-yl icos-11-enoate (295 mg, 1 equiv., 0.500 mmol) was measured in under nitrogen. Next, the solution of the catalyst in benzene-d6 [(2-bromo-6-fluorophenyl)bis(2,3,6-trifluorophenyl)borane (Compound 2) (2.23 mg, 100 μL, 0.05 M in benzene-d6, 0.01 equiv., 5.00 μmol)] was added at room temperature. Then, under stirring, triethylsilane (64.0 mg, 87.8 μL, 1.1 equiv., 550 μmol) was added dropwise to the reaction mixture. The reaction was stirred overnight. The reaction went to complete conversion, as judged by ¹H NMR.

Triethyl((1-(icos-11-en-1-yloxy)icos-11-en-1-yl)oxy)silane: ¹H NMR (500 MHz, Benzene-d6) δ 5.51 (ddt, J=5.9, 4.4, 1.7 Hz, 4H), 4.90 (dd, J=6.0, 4.4 Hz, 1H), 3.75 (dtd, J=8.2, 6.5, 1.5 Hz, 1H), 3.41 (dtd, J=7.9, 6.5, 1.3 Hz, 1H), 2.12 (tdd, J=7.4, 5.5, 2.5 Hz, 8H), 1.89-1.23 (m, 56H), 1.08 (t, J=7.9 Hz, 9H), 0.92 (t,J=6.8 Hz, 6H), 0.71 (q, J=8.0 Hz, 6H).

Next, the silyl-acetal was hydrolysed by diluting the reaction mixture with 5 mL of THF and adding aqueous hydrochloric acid (182 mg, 5.0 mL, 1 M, 10 equiv., 5.0 mmol) to it. After 16 hours, the reaction mixture was extracted with 30 mL ethyl-acetate, washed with 30 mL saturated NaHCO₃ solution, dried over MgSO₄ and the solvent was removed under reduced pressure. The resulting oil contained icos-11-enal as a major constituent (>55 m/m %) and also minor contaminants from 11-eicosanol (<40 m/m %) and hexaethyldisiloxane (<5 m/m %) based on ¹H NMR. The aldehydic proton is clearly visible at δ 9.35 (t, J=1.8 Hz, 1H), while the peaks of the silyl-acetal disappeared completely, indicating total conversion. The double bonds remained intact throughout the process as indicated by the olefinic H peaks (Benzene-d6) δ 5.48 (t, J=5.0 Hz, 2H).

Icos-11-enal: ¹H NMR (500 MHz, Benzene-d6) δ 9.35 (t, J=1.8 Hz, 1H), 5.48 (t, J=5.0 Hz, 2H), 2.09 (q, J=6.6 Hz, 4H), 1.85 (td, J=7.3, 1.9 Hz, 2H), 1.45 — 1.04 (m, 26H), 0.90 (t, J=6.8 Hz, 3H).

### Example 11

### Reduction of Ethyl 2,2,2-trifluoroacetate

2,2,2-trifluoroacetaldehyde is an important synthetic building block in medicinal chemistry. The synthesis and usage of this compound on the other hand is cumbersome due to its low boiling point (20° C.). The use of its silyl-acetal as a synthetic precursor could become a viable alternative.

The silyl-acetal can be synthesized starting from the widely available ethyl 2,2,2-trifluoroacetate, but due to the low Lewis basicity of this ester, a stronger Lewis acid is needed, like the F₉(F₄), borane (Compound 7).

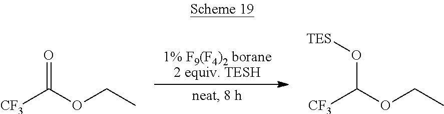

In an oven dried 4 mL vial the ester, ethyl 2,2,2-trifluoroacetate (71.0 mg, 59.5 μL, 1 equiv., 0.500 mmol) was measured in under nitrogen. Next, the solution of the catalyst in benzene-d6 (perfluoro-[1,1′-biphenyl]-2-yl)bis(2,3,5,6-tetrafluorophenyl)borane (Compound 7, Entry 18) (2.68 mg, 100 μL, 0.05 M in benzene-d6, 0.01 equiv., 5.00 μmol) was added at room temperature. Then, under stirring, triethylsilane (116 mg, 160 μL, 2 equiv., 1.00 mmol) was added dropwise to the reaction mixture. The reaction was stirred overnight. The reaction went to complete conversion, as judged by ¹H NMR.

Product: (1-ethoxy-2,2,2-trifluoroethoxy)triethylsilane (90% NMR yield)

### ¹H NMR

Partial ¹H NMR (500 MHz, Benzene-d6) δ 4.78 (q, J=3.8 Hz, 1H), 3.53-3.45 (m, 1H), 3.30-3.37 (m, 1H).

### Example 12

### Reduction of γ-Butyrolactone

The selective reduction of lactones into lactols is a difficult synthetic problem often encountered during the syntheses of some important pharmaceutical intermediates. This is also the case in the synthesis of prostaglandins, where the reduction of a y-butyrolactone moiety is a challenging task. The problem of overreduction in the case of lactones is even more pronounced, so a weaker Lewis acid is needed. This concept is demonstrated in this example, where we used a borane having weaker Lewis acidity, the BrF(F₃s)₂ borane (Compound 5), to reduce γ-butyrolactone with high selectivity.

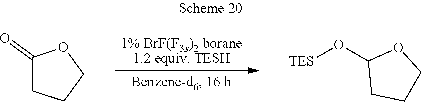

In an oven dried 4 mL vial the lactone, dihydrofuran-2(3H)-one (86 mg, 76 μL, 1 equiv., 1.0 mmol) was measured in under nitrogen, and dissolved in 0.8 ml benzene-d6. Next, the solution of the catalyst in benzene-d6 (2-bromo-6-fluorophenyl)bis(2,4,6-trifluorophenyl)borane (Compound 5, Entry 19) (4.4 mg, 0.20 mL, 0.05 M in benzene-d6, 0.01 equiv., 10 μmol) was added at room temperature. Then, under stirring, triethylsilane (0.14 g, 0.19 mL, 1.2 equiv., 1.2 mmol) was added dropwise to the reaction mixture. The reaction was stirred for a further 16 hours at room temperature. Complete conversion was achieved, as judged by ¹H NMR, and the amount of overreduced side product (3,3,10,10-tetraethyl-4,9-dioxa-3,10-disiladodecane) was minimal (<12 m/m %).

Product: triethyl((tetrahydrofuran-2-yl)oxy)silane (86% NMR yield)

### ¹H NMR

¹H NMR (500 MHz, Chloroform-d) δ 5.57 (t, J=2.6 Hz, 1H), 4.06 (td, J=8.2, 4.7 Hz, 1H), 3.88-3.82 (m, 1H), 2.14-2.04 (m, 1H), 1.94-1.82 (m, 3H), 1.04 (t, J=7.9 Hz, 12H), 0.70 (q, J=8.0 Hz, 7H).

### Example 13

### Reduction of Methyl 3-phenylpropanoate

General method for the hydrosilylation of methyl 3-phenylpropanoate ester using catalysts according to the present invention (Scheme 1, Table 1)

The reaction was performed under inert conditions, neat or using dried solvents. Importantly, the catalyst can operate even in the presence of small amounts of water (technical grade solvents).

In an oven dried 20 mL vial the ester, methyl 3-phenylpropanoate substrate (0.82 g, 0.79 mL, 1 equiv., 5.0 mmol) was measured in and dissolved in 9 mL abs. toluene. Next, the solution of the catalyst in toluene ((2-bromo-6-fluorophenyl)bis(2,3,6-trifluorophenyl)borane (Compound 2, Entry 16) (23 mg, 1.0 mL, 0.05 M in toluene, 0.01 equiv., 50 μmol)) was added at room temperature. Then, under stirring, triethylsilane (0.64 g, 0.88 mL, 1.1 equiv., 5.5 mmol) was added dropwise to the reaction mixture. Soon, the reaction started to warm up and the evolution of small amounts of hydrogen gas is observed (from trace amounts of water/alcohols/carboxylic acid). The reaction was further stirred at room temperature for 16 hours, until the end of the conversion of the ester, as judged by NMR or GC-MS. Next, the reaction mixture was passed through a short pad of silica and eluted with hexanes. The filtrate was concentrated in vacuo to obtain the product triethyl(1-methoxy-3-phenylpropoxy)silane (1.39 g, 4.95 mmol, 99% yield).

The above process can be applied with the use of the other catalysts compound given in Table 1., with the necessary modifications being within the general knowledge of a skilled person.

### ¹H NMR

¹H NMR (500 MHz, Benzene-d6) δ 7.18-7.12 (m, 5H), 4.70 (dd, J=5.9, 4.3 Hz, 1H), 3.19 (s, 3H), 2.78-2.73 (m, 2H), 2.06-1.98 (m, 1H), 1.97-1.89 (m, 1H), 1.00 (t, J=7.9 Hz, 9H), 0.61 (q, J=8.0 Hz, 6H).

### ¹³C NMR

¹³C NMR (126 MHz, Benzene-d6) δ 142.4 (1C), 128.8 (2C), 128.7 (2C), 126.1 (1C), 98.7 (1C), 53.2 (1C), 39.3 (1C), 31.2 (1C), 7.1 (3C), 5.6 (3C).

### Example 14

### Reduction of Ethyl 4-bromobutanoate

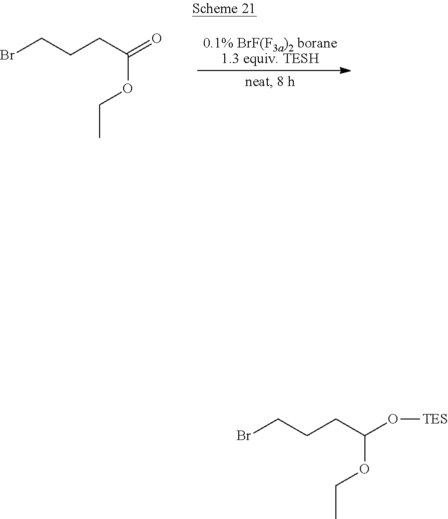

In an oven dried 20 mL vial the ester, ethyl 4-bromobutanoate (2.93 g, 2.15 mL, 1 equiv., 15.0 mmol) was measured in under nitrogen. Next, the solution of the catalyst in benzene-d6 (2-bromo-6-fluorophenyl)bis(2,3,6-trifluorophenyl)borane (Compound 2) (6.70 mg, 300 μL, 0.05 M in benzene-d6, 0.001 equiv., 15.0 μmol) was added at room temperature. Then, under stirring, triethylsilane (2.27 g, 3.1 mL, 1.3 equiv., 19.5 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: (4-bromo-1-ethoxybutoxy)triethylsilane (4.54 g, 14.6 mmol, 97% yield).

### ¹H NMR

¹H NMR (500 MHz, Chloroform-d) δ 4.81 (t, J=5.0 Hz, 1H), 3.70 (dq, J=9.2, 7.1 Hz, 1H), 3.46-3.36 (m, 3H), 2.03-1.89 (m, 2H), 1.78-1.67 (m, 2H), 1.19 (t, J=7.0 Hz, 3H), 0.98 (t, J=7.9 Hz, 9H), 0.64 (q, J=8.0 Hz, 6H).

### Example 15

### Reduction of Ethyl 3-bromopropanoate

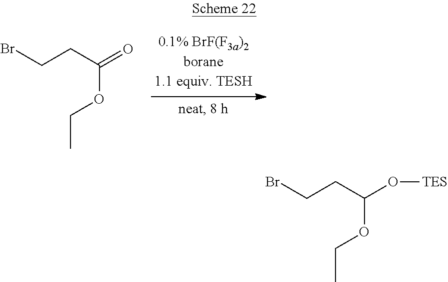

In an oven dried 20 mL vial the ester, ethyl 3-bromopropanoate (3.62 g, 2.55 mL, 1 equiv., 20.0 mmol) was measured in under nitrogen. Next, the solution of the catalyst in benzene-d6 (2-bromo-6-fluorophenyl)bis(2,3,6-trifluorophenyl)borane (Compound 2) (8.94 mg, 400 μL, 0.05 M In benzene-d6, 0.001 equiv., 20.0 μmol) was added at room temperature. Then, under stirring, triethylsilane (2.56 g, 3.51 mL, 1.1 equiv., 22.0 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: (3-bromo-1-ethoxypropoxy)triethylsilane (6.2 g, 21 mmol, 99+% yield).

### ¹H NMR

¹H NMR (300 MHz, Benzene-d6) δ 4.92 (dd, J=5.7, 4.4 Hz, 1H), 3.56 (dq, J=9.1, 7.1 Hz, 1H), 3.35-3.15 (m, 3H), 2.11-1.90 (m, 2H), 1.06 (t, J=7.0 Hz, 3H), 0.98 (t, J=7.9 Hz, 9H), 0.66-0.54 (m, 6H).

### Example 16

### Reduction of Ethyl 5-bromopentanoate

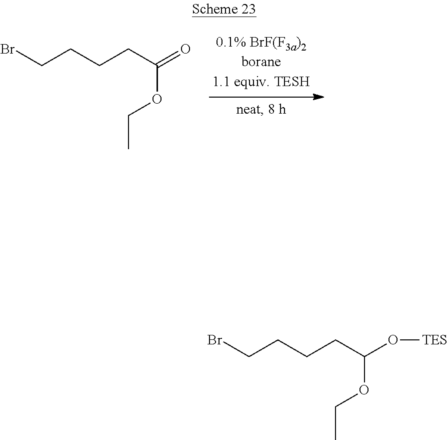

In an oven dried 20 mL vial the ester, ethyl 5-bromopentanoate (4.18 g, 3.17 mL, 1 equiv., 20.0 mmol) was measured in under nitrogen. Next, the solution of the catalyst in benzene-d6 (2-bromo-6-fluorophenyl)bis(2,3,6-trifluorophenyl)borane (Compound 2) (8.94 mg, 400 μL, 0.05 M in benzene-d6, 0.001 equiv., 20.0 μmol) was added at room temperature. Then, under stirring, triethylsilane (2.56 g, 3.51 mL, 1.1 equiv., 22.0 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: ((5-bromo-1-ethoxypentyl)oxy)triethylsilane (6.5 g, 20 mmol, 99+% yield).

### ¹H NMR

¹H NMR (500 MHz, Benzene-d6) δ 4.71 (dd, J=5.8, 4.1 Hz, 1H), 3.62 (dq, J=9.1, 7.1 Hz, 1H), 3.27 (dq, J=9.1, 7.0 Hz, 1H), 2.97 (t, J=6.7 Hz, 2H), 1.61-1.38 (m, 6H), 1.12 (t, J=7.0 Hz, 3H), 1.02 (t, J=8.0 Hz, 9H), 0.64 (q, J=8.0 Hz, 6H).

### Example 17

### Reduction of Ethyl 6-bromohexanoate

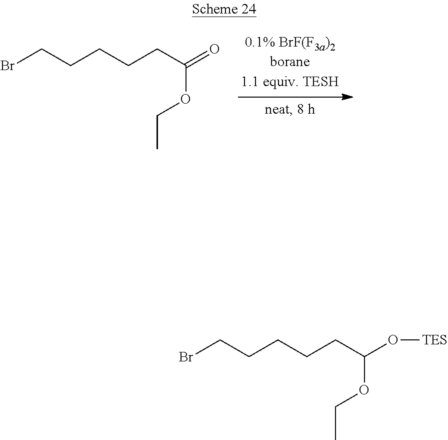

In an oven dried 20 mL vial the ester, ethyl 6-bromohexanoate (3.35 g, 2.67 mL, 1 equiv., 15.0 mmol) was measured in under nitrogen. Next, the solution of the catalyst in benzene-d6 (2-bromo-6-fluorophenyl)bis(2,3,6-trifluorophenyl)borane (Compound 2) (6.70 mg, 300 μL, 0.05 M in benzene-d6, 0.001 equiv., 15.0 μmol was added at room temperature. Then, under stirring, triethylsilane (1.92 g, 2.64 mL, 1.1 equiv., 16.5 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: ((6-bromo-1-ethoxyhexyl)oxy)triethylsilane (4.9 g, 14 mmol, 96% yield).

### ¹H NMR

¹H NMR (500 MHz, Benzene-d6) δ 4.76 (dd, J=5.8, 4.4 Hz, 1H), 3.65 (dq, J=9.1, 7.1 Hz, 1H), 3.32 (dq, J=9.1, 7.0 Hz, 1H), 2.95 (t, J=6.8 Hz, 2H), 1.67-1.47 (m, 4H), 1.37-1.28 (m, 2H), 1.21 (q, J=7.6 Hz, 2H), 1.15 (t, J=7.0 Hz, 3H), 1.04 (t, J=7.9 Hz, 9H), 0.66 (q, J=8.0 Hz, 6H).

### Example 18

### Reduction of Isopropyl 4-Bromobutanoate

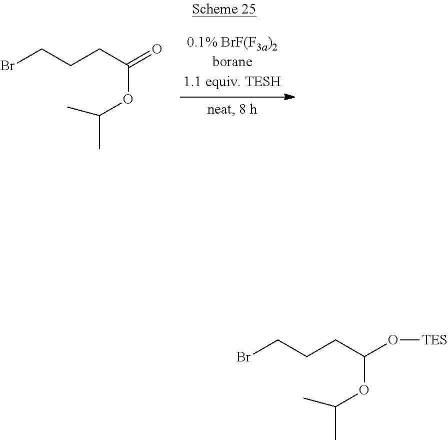

In an oven dried 20 mL vial the ester, isopropyl 4-bromobutanoate (2.09 g, 1 equiv., 10.0 mmol) was measured in under nitrogen. Next, the solution of the catalyst in benzene-d6 (2-bromo-6-fluorophenyl)bis(2,3,6-trifluorophenyl)borane (Compound 2) (4.47 mg, 200 μL, 0.05 M in benzene-d6, 0.001 equiv., 10.0 μmol) was added at room temperature. Then, under stirring, triethylsilane (1.28 g, 1.76 mL, 1.1 equiv., 11.0 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: (4-bromo-1-isopropoxybutoxy)triethylsilane (2.84 g, 8.73 mmol, 87% yield).

### ¹H NMR

¹H NMR (500 MHz, Benzene-d6) δ 4.80 (dd, J=5.6, 4.1 Hz, 1H), 3.75 (hept, J=6.2 Hz, 1H), 3.13-3.02 (m, 2H), 1.91-1.75 (m, 2H), 1.70-1.60 (m, 2H), 1.14 (d, J=6.2 Hz, 3H), 1.04-0.95 (m, 12H), 0.62 (q, J=7.7 Hz, 6H).

### Example 19

### Reduction of Ethyl 2-(2-chloroethoxy)acetate

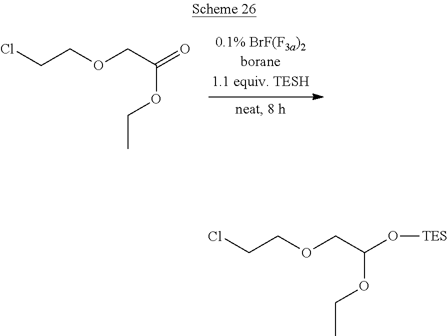

In an oven dried 20 mL vial the ester, ethyl 2-(2-chloroethoxy)acetate (1.67 g, 1 equiv., 10.0 mmol) was measured in under nitrogen. Next, the solution of the catalyst in benzene-d6 (2-bromo-6-fluorophenyl)bis(2,3,6-trifluorophenyl)borane (Compound 2) (4.47 mg, 200 μL, 0.05 M in benzene-d6, 0.001 equiv., 10.0 μmol) was added at room temperature. Then, under stirring, triethylsilane (1.28 g, 1.76 mL, 1.1 equiv., 11.0 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: (2-(2-chloroethoxy)-1-ethoxyethoxy)triethylsilane (2.5 g, 8.8 mmol, 88% yield).

### ¹H NMR

¹H NMR (300 MHz, Benzene-d6) δ 4.91 (t, J=4.9 Hz, 1H), 3.74-3.61 (m, 1H), 3.44-3.27 (m, 5H), 3.20-3.14 (m, 2H), 1.12 (td, J=7.1, 1.2 Hz, 3H), 1.02 (t, J=7.8 Hz, 9H), 0.65 (q, J=8.3 Hz, 6H).

### Example 20

Reduction of Methyl 2-bromopropanoate

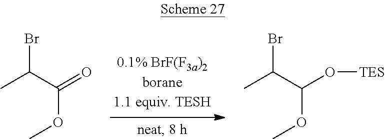

In an oven dried 20 mL vial the ester, methyl 2-bromopropanoate (1.67 g, 1.12 mL, 1 equiv., 10.0 mmol) was measured in under nitrogen. Next, the solution of the catalyst in benzene-d6 (2-bromo-6-fluorophenyl)bis(2,3,6-trifluorophenyl)borane (Compound 2) (4.47 mg, 200.0 μL, 0.05 M in benzene-d6, 0.001 equiv., 10.0 μmol) was added at room temperature. Then, under stirring, triethylsilane (1.28 g, 1.76 mL, 1.1 equiv., 11.0 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain (2-bromo-1-methoxypropoxy)triethylsilane (2.7 g, 9.5 mmol, 95% yield). The product is a mixture of the possible diastereomers in a 3:1 ratio.

### ¹H NMR

### Major Diastereomer:

¹H NMR (300 MHz, Chloroform-d) δ 4.71 (d, J=4.6 Hz, 1H), 4.08-3.96 (m, 1H), 3.38 (d, J=0.7 Hz, 3H), 1.64 (d, J=6.7 Hz, 3H), 1.05-0.94 (m, 9H), 0.69 (q, J=7.8 Hz, 6H).

### Minor Diastereomer:

¹H NMR (300 MHz, Chloroform-d) δ 4.76 (d, J=3.8 Hz, 1H), 4.00-3.85 (m, 1H), 3.40 (d, J=0.8 Hz, 3H), 1.65 (d, J=6.9 Hz, 3H), 1.00 (t, J=7.9 Hz, 9H), 0.68 (q, J=7.9 Hz, 6H).

### Example 21

### Reduction of Ethyl 2-(2-bromoethoxy)acetate

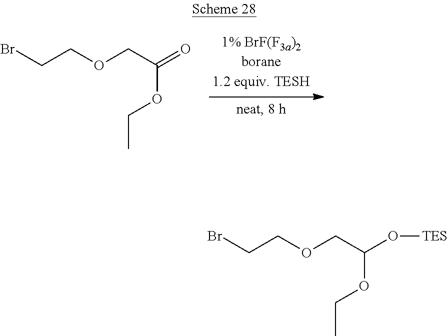

In an oven dried 20 mL vial the ester, ethyl 2-(2-bromoethoxy)acetate (2.11 g, 1 equiv., 10.0 mmol) was measured in under nitrogen. Next, the solution of the catalyst in benzene-d6 (2-bromo-6-fluorophenyl)bis(2,3,6-trifluorophenyl)borane (Compound 2) (44.7 mg, 2.00 mL, 0.05 M in benzene-d6, 0.01 equiv., 100 μmol) was added at room temperature. Then, under stirring, triethylsilane (1.40 g, 1.92 mL, 1.2 equiv., 12.0 mmol) was added dropwise to the reaction mixture. The reaction was stirred overnight. The reaction went to complete conversion, as judged by ¹H NMR. Next day, the reaction mixture was passed through a short pad of silica and eluted with hexanes. The filtrate was concentrated in vacuo to obtain the product: (2-(2-bromoethoxy)-1-ethoxyethoxy)triethylsilane (3.2 g, 9.8 mmol, 98% yield).

### ¹H NMR

¹H NMR (500 MHz, Benzene-d6) δ 4.91 (t, J=4.9 Hz, 1H), 3.67 (dq, J=9.2, 7.1 Hz, 1H), 3.44 -3.32 (m, 5H), 3.01 (t, J=6.1 Hz, 2H), 1.13 (t, J=7.0 Hz, 3H), 1.02 (t, J=7.8 Hz, 9H), 0.65 (qd, J=7.9, 1.4 Hz, 6H).

### Example 22

### Reduction of Ethyl 4-bromo-2-fluorobutanoate

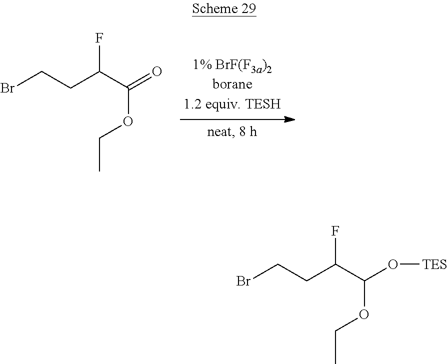

In an oven dried 4 mL vial the ester, ethyl 4-bromo-2-fluorobutanoate (533 mg, 1 equiv., 2.50 mmol) was measured in under nitrogen. Next, the solution of the catalyst in benzene-d6 (2-bromo-6-fluorophenyl)bis(2,3,6-trifluorophenyl)borane (Compound 2) (11.2 mg, 500 μL, 0.05 M in benzene-d6, 0.01 equiv., 25.0 μmol) was added at room temperature. Then, under stirring, triethylsilane (349 mg, 479 μL, 1.2 equiv., 3.00 mmol) was added dropwise to the reaction mixture. The reaction was stirred overnight. The reaction went to complete conversion, as judged by ¹H NMR. Next day, the reaction mixture was passed through a short pad of silica and eluted with hexanes. The filtrate was concentrated in vacuo to obtain the product: (4-bromo-1-ethoxy-2-fluorobutoxy)triethylsilane (720 mg, 2.19 mmol, 88% yield).

### ¹H NMR

¹H NMR (500 MHz, Chloroform-d) δ 4.89 (dd, J=6.9, 3.7 Hz, 1H), 4.62-4.48 (m, 1H), 3.75 (dq, J=9.2, 7.1 Hz, 1H), 3.60-3.48 (m, 3H), 2.31-2.16 (m, 2H), 1.22 (t, J=7.0 Hz, 3H), 1.00 (t, J=7.9 Hz, 9H), 0.68 (q, J=8.1 Hz, 6H).

### Example 23

### Reduction of Ethyl decanoate

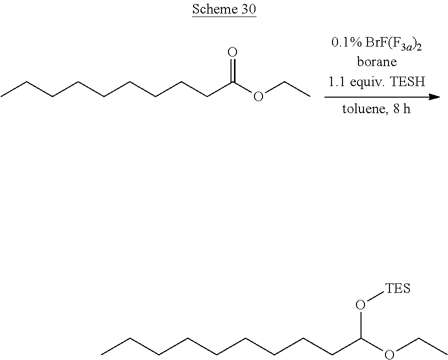

In an oven dried 500 mL flask the ester, ethyl decanoate (40.0 g, 1 equiv., 200 mmol) was measured in under nitrogen and dissolved in 200 ml dry toluene. Next, the solution of the catalyst in toluene (2-bromo-6-fluorophenyl)bis(2,3,6-trifluorophenyl)borane (Compound 2) (89.2 mg, 3.99 mL, 0.05 M in toluene, 0.001 equiv., 200 μmol) was added at room temperature. Then, under stirring triethylsilane (25.5 g, 35.1 mL, 1.1 equiv., 220 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: ((1-ethoxydecyl)oxy)triethylsilane (62 g, 0.20 mol, 98% yield)

### ¹H NMR

¹H NMR (500 MHz, Chloroform-d) δ 4.75 (dd, J=6.2, 4.4 Hz, 1H), 3.69 (dq, J=9.1, 7.1 Hz, 1H), 3.41 (dq, J=9.1, 7.0 Hz, 1H), 1.66 -1.48 (m, 2H), 1.40-1.23 (m, 14H), 1.19 (t, J=7.0 Hz, 3H), 0.98 (t, J=8.0 Hz, 9H), 0.88 (t, J=6.9 Hz, 3H), 0.64 (q, J=7.9 Hz, 6H).

### Example 23

### Reduction of Ethyl 4-bromopentanoate

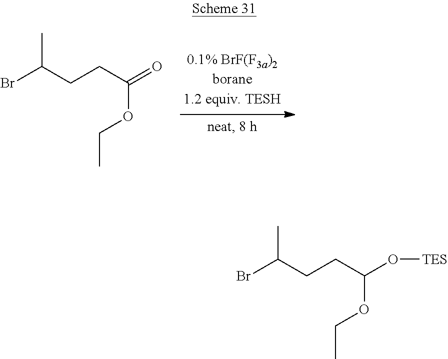

In an oven dried 4 mL vial the ester, ethyl 4-bromopentanoate (1.05 g, 1 equiv., 5.00 mmol) was measured in under nitrogen. Next, the solution of the catalyst in toluene (2-bromo-6-fluorophenyl)bis(2,3,6-trifluorophenyl)borane (Compound 2) (2.23 mg, 100 μL, 0.05 M in toluene, 0.001 equiv., 5.00 μmol) was added at room temperature. Then, under stirring, triethylsilane (698 mg, 958 μL, 1.2 equiv., 6.00 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: ((4-bromo-1-ethoxypentyl)oxy)triethylsilane (1.6 g, 4.9 mmol, 98% yield).

### ¹H NMR

¹H NMR (500 MHz, Chloroform-d) δ 4.80 (ddd, J=14.8, 5.9, 4.0 Hz, 1H), 4.21-4.11 (m, 1H), 3.70 (dqd, J=9.1, 7.1, 1.9 Hz, 1H), 3.40 (dqd, J=9.2, 7.0, 1.2 Hz, 1H), 1.97 -1.76 (m, 4H), 1.71 (d, J=6.7 Hz, 3H), 1.19 (t, J=7.1 Hz, 3H), 1.03-0.95 (m, 9H), 0.66 (td, J=7.9, 1.7 Hz, 6H).

### Example 24

### Reduction of Ethyl 4-bromo-2,2-difluorobutanoate

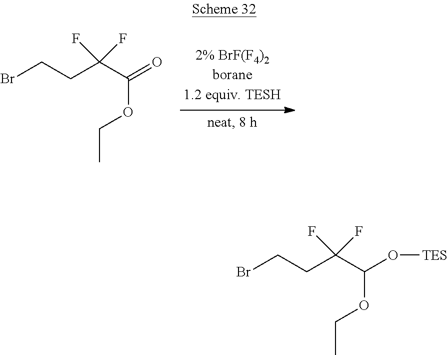

In an oven dried 4 mL vial the ester, ethyl 4-bromo-2,2-difluorobutanoate (578 mg, 1 equiv., 2.50 mmol) was measured in under nitrogen. Next, the solution of the catalyst in toluene (2-bromo-6-fluorophenyl)bis(2,3,5,6-tetrafluorophenyl)borane (Compound 1) (24.1 mg, 1.00 mL, 0.05 M in toluene, 0.02 equiv., 50.0 μmol was added at room temperature. Then, under stirring, triethylsilane (349 mg, 479 μL, 1.2 equiv., 3.00 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain the product: (4-bromo-1-ethoxy-2,2-difluorobutoxy)triethylsilane (448 mg, 1.29 mmol, 52% yield).

### ¹H NMR

¹H NMR (500 MHz, Chloroform-d) δ 4.73 (dd, J=4.6, 3.8 Hz, 1H), 3.75 (dq, J=9.2, 7.1 Hz, 1H), 3.58-3.47 (m, 3H), 2.62-2.50 (m, 2H), 1.23 (t, J=7.0 Hz, 3H), 0.98 (t, J=8.0 Hz, 9H), 0.70-0.64 (m, 6H).

### Example 25

Reduction of Ethyl 4-bromo-2-methylbutanoate

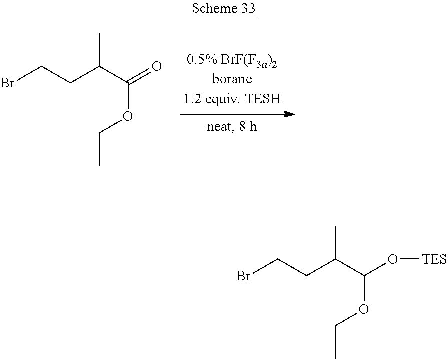

In an oven dried 4 mL vial the ester, ethyl 4-bromo-2-methylbutanoate (1.05 g, 1 equiv., 5.00 mmol) was measured in under nitrogen. Next, the solution of the catalyst in benzene-d6 (2-bromo-6-fluorophenyl)bis(2,3,6-trifluorophenyl)borane (Compound 2) (11.2 mg, 500 0.05 M in benzene-d6, 0.005 equiv., 25.0 μmol) was added at room temperature. Then, under stirring, triethylsilane (698 mg, 958 1.2 equiv., 6.00 mmol) was added dropwise to the reaction mixture. The reaction was stirred overnight. The reaction went to complete conversion, as judged by 1H NMR. Next day, the reaction mixture was passed through a short pad of silica and eluted with hexanes. The filtrate was concentrated in vacuo to obtain (4-bromo-1-ethoxy-2-methylbutoxy)triethylsilane (1.48 g, 4.55 mmol, 91% yield). The product is a mixture of the possible diastereomers in a 3:2 ratio.

¹H NMR

Major diastereomer:

1H NMR (500 MHz, Chloroform-d) δ 4.62 (d, J=3.7 Hz, 1H), 3.72-3.64 (m, 1H), 3.57-3.50 (m, 1H), 3.46-3.38 (m, 2H), 2.06 (dtd, J=14.2, 7.7, 5.1 Hz, 1H), 1.78 -1.67 (m, 2H), 1.19 (t, J=7.0 Hz, 3H), 0.99 (t, J=8.0 Hz, 9H), 0.94 (d, J=6.8 Hz, 3H), 0.65 (q, J=8.0 Hz, 6H).

Minor diastereomer:
¹H NMR (500 MHz, Chloroform-d) δ 4.59 (d, J=4.1 Hz, 1H), 3.72-3.64 (m, 1H), 3.57-3.50 (m, 1H), 3.46-3.38 (m, 2H), 2.16 (dtd, J=14.1, 8.1, 4.6 Hz, 1H), 1.90-1.80 (m, 2H), 1.19 (t, J=7.0 Hz, 3H), 0.99 (t, J=8.0 Hz, 9H), 0.91 (d, J=6.9 Hz, 3H), 0.65 (q, J=8.0 Hz, 6H).

### Example 26

### Synthesis of (2-bromo-6-fluorophenyl)bis(2,6-difluorophenyl)borane (Compound 9, See Entry 20)

The compound was prepared as described below and illustrated in schemes 3, 4 and 34.

Step a) and Step b) are analogues to EXAMPLE 1.

Step c) Synthesis of (2-bromo-6-fluorophenyl)bis(2,6-difluorophenyl)Borane (Compound 9)

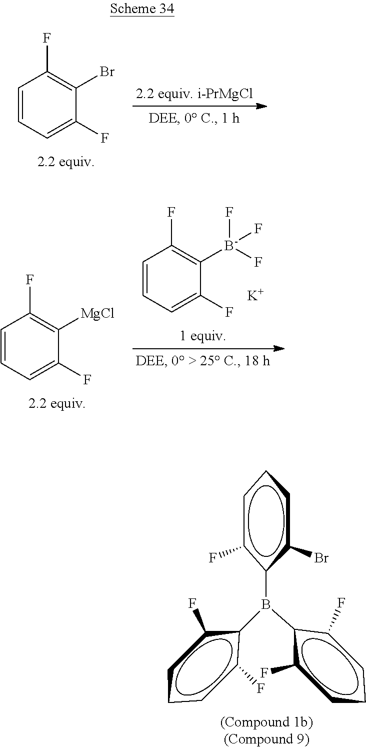

A 50 mL 3-necked flask was equipped with a reflux condenser and N₂ inlet, Magnesium turnings (0.95 g, 2.2 equiv., 39.2 mmol) were measured in and activated with iodine. Then, 15 mL abs. diethyl ether was added followed by the dropwise addition of 2-chloropropane (3.08 g, 3.57 mL, 2.2 equiv., 39.2 mmol). The solution started to warm up and reflux. Additional 15 mL diethyl ether was added, and dropwise addition of 2-chloropropane was continued to maintain the reflux. In another 250 mL 2-necked flask, 2-bromo-1,3-difluorobenzene (7.56 g, 4.42 mL, 2.2 equiv., 39.2 mmol) was measured in and dissolved in 60 mL abs. diethyl ether, after which it was cooled to 0° C. The previously prepared Grignard solution was added dropwise via syringe in 45 min, keeping the reaction temperature below 5° C. After completion of the addition, the reaction mixture was stirred for 1 h. In a 250 mL Schlenk flask, potassium (2-bromo-6-fluorophenyl)trifluoroborate (Compound 1b) (5.00 g, 1 equiv., 17.8 mmol) was measured in under N₂ suspended in 10 mL abs. diethyl ether and cooled down to −78° C. The cool (−78° C.) Grignard solution was added via cannula within 20 min, while keeping the temperature under −60° C. The reaction mixture was left to warm up to 25° C. and was stirred for an additional 18h. Afterwards, the solvent was evaporated at 50° C. in vacuo. Next, 60 mL abs. toluene was added, and the suspension was sonicated for 10 minutes. with the resulting precipitate was filtered off and washed with 2×10 mL abs. toluene. The combined filtrate was then evaporated at 70° C. in vacuo, resulting an off-white solid. Then, 20 mL abs. hexane was added, and the resulting suspension was filtered at −78° C. to yield the product as a white crystalline powder (2.92 g, 7.10 mmol, 40% yield).

### ¹H NMR

¹H NMR (500 MHz, Benzene-d6) δ 7.04 (dd, J=7.8, 1.0 Hz, 1H), 6.73 (tt, J=8.3, 6.5 Hz, 2H), 6.66-6.55 (m, 2H), 6.43 (t, J=8.1 Hz, 4H). ¹⁹F NMR

¹⁹ F NMR (282 MHz, Benzene-d6) δ −97.70 (t, J=7.1 Hz, 4F), −102.58-−102.69 (m, 1F).

### ¹³ C NMR

Partial ¹³C NMR (126 MHz, Benzene-d6) δ 166.2 (dd, J=253.3, 11.1 Hz, 4C), 162.9 (d, J=244.5 Hz, 1C), 136.1 (t, J=11.5 Hz, 2C), 131.8 (d, J=9.1 Hz, 1C), 128.1 (d, J=3.2 Hz, 1C), 123.8 (d, J=10.1 Hz, 1C), 113.8 (d, J=23.8 Hz, 1C), 111.6-111.4 (m, 4C).

### Example 27

### Alternative Reduction of ethyl 4-bromobutanoate using TMDS

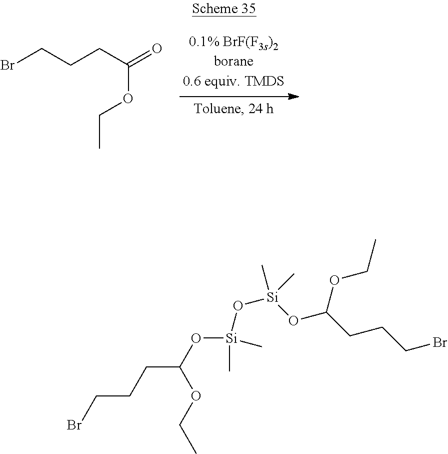

In an oven dried 20 mL vial the ester, ethyl 4-bromobutanoate (1.29 g, 0.95 mL, 1 equiv., 6.60 mmol) was measured in under nitrogen and dissolved in 6.6 ml abs. toluene. Next, the solution of the catalyst in benzene-d6 (2-bromo-6-fluorophenyl)bis(2,4,6-trifluorophenyl)borane (Compound 5) (2.95 mg, 132 μL, 0.05 M in benzene-d6, 0.001 equiv., 6.60 μmol) was added at room temperature. Then, under stirring, 1,1,3,3-tetramethyldisiloxane (TMDS) (532 mg, 0.70 mL, 0.6 equiv., 3.96 mmol) was added dropwise to the reaction mixture. The reaction was stirred overnight. The reaction went to complete conversion, as judged by 1H NMR. Next day, the solvents were evaporated, and the crude product was purified using flash chromatography on silica gel with hexanes/ethyl acetate gradient elution. After chromatography, the fractions containing the product were concentrated in vacuo to give the product 4,10-bis(3-bromopropyl)-6,6,8,8-tetramethyl-3,5,7,9,11-pentaoxa-6,8-disilatridecane (1.64 g, 3.13 mmol, 95% yield).

### ¹H NMR

### ¹H NMR (500 MHz, Chloroform-d) δ 4.88 (t, J=5.1 Hz, 2H), 3.77-3.68 (m, 2H), 3.47-3.35 (m, 6H), 2.02 -1.91 (m, 4H), 1.79-1.72 (m, 4H), 1.20 (t, J=7.1 Hz, 6H), 0.19-0.16 (m, 12H).

### Example 28

Reduction of Ethyl 3-phenylpropanoate using TMDS

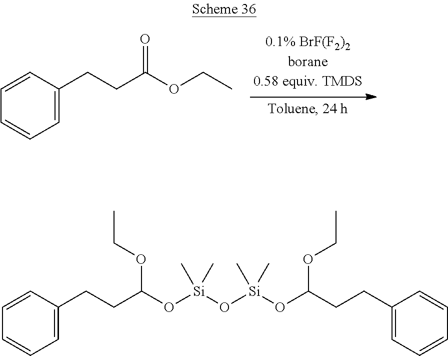 [SMILES: CCOC(=O)CCc1ccccc1 ; CCOC(CCc1ccccc1)O[Si](C)(C)O[Si](C)(C)OC(CCc1ccccc1)OCC]

In an oven dried 20 mL vial the ester, ethyl 3-phenylpropanoate (1.07 g, 1 equiv., 6.00 mmol) was measured in under nitrogen and dissolved in 6.0 ml abs. toluene. Next, the solution of the catalyst in benzene-d6 (2-bromo-6-fluorophenyl)bis(2,6-difluorophenyl)borane (Compound 9) (2.47 mg, 120 μL, 0.05 M in benzene-d6, 0.001 equiv., 6.00 μmol) was added at room temperature. Then, under stirring, 1,1,3,3-tetramethyldisiloxane (TMDS) (467 mg, 0.62 mL, 0.58 equiv., 3.48 mmol) was added dropwise to the reaction mixture. The reaction was stirred overnight. The reaction went to complete conversion, as judged by 1H NMR. Next day, the solvents were evaporated, and the crude product was purified using flash chromatography on silica gel with hexanes/ethyl acetate gradient elution. After chromatography, the fractions containing the product were concentrated in vacuo to give the product 6,6,8,8-tetramethyl-4,10-diphenethyl-3,5,7,9,11-pentaoxa-6,8-disilatridecane (1.15 g, 2.33 mmol, 78% yield).

### ¹H NMR

¹H NMR (500 MHz, Chloroform-d) δ 7.29-7.24 (m, 4H), 7.20-7.15 (m, 6H), 4.83 (dd, J=6.1, 4.4 Hz, 2H), 3.74 (dq, J=9.3, 7.1 Hz, 2H), 3.39 (dq, J=9.2, 7.0 Hz, 2H), 2.76 — 2.62 (m, 4H), 2.01 — 1.84 (m, 4H), 1.21 (td, J=7.0, 1.3 Hz, 6H), 0.13 (d, J=4.1 Hz, 12H).

General Remarks

During the evaluation/explanation of the results, the following theoretical assumptions can be made. At first, the steric factor should be taken into consideration, because the ortho-substituents on the aryl rings significantly inhibit the access to the boron center. Thus, the principle of size exclusion is realized, the essence of which is that the boranes do not form stable adducts with the Lewis basic components present in the reaction mixture, but the triethylsilane still has access to them. This improves the selectivity, although significant steric “congestion” may lead to a decrease in the reactivity. Another important factor is the Lewis acidity of boranes. Increasing this also increases the reactivity to a certain level, but beyond this level, the electron-withdrawing substituents excessively stabilize the forming hydride intermediate, thereby reducing its reactivity. However, increasing the reactivity of boranes can also reduce the selectivity. A further aspect is the reactivity of the substrate (ester or lactone) to be reduced. In case of a reactive substrate a less reactive catalyst of the present invention can be proper and vice versa. The selection of the proper catalyst to a specific substrate needs a “fine-tuning” of the substituent pattern of the catalyst (increasing or decreasing the Lewis acid character of it by the use of the substituents providing the desired electron withdrawing effect). The theoretical selection can be made on the basis of the expectable knowledge of a skilled person and the success of the selected substituent pattern can be checked by relatively simple experiments, i.e. without undue burden on the skilled person working on this filed. This is a very important feature of the present invention which allows a general use of the invented catalyst family.

1. A compound of general formula (I) 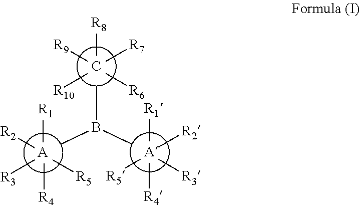
wherein
B is boron;
A ring and A′ ring, independently from each other, are aryl or heteroaryl groups, wherein
R₁ and R′₁ are independently selected from the group consisting of H, D and F;
R₅ and R′₅ are independently selected from the group consisting of H, D and F;
each R₂, R₃, R₄, R′₂, R′₃ and R′₄ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
C ring is aryl group, wherein
R₆ is selected from the group consisting of H, D and F;
R₁₀ is selected from the group consisting of Cl, Br, I, SF₅, alkyl, cycloalkyl, alkenyl, cycloalkenyl, phenyl, heteroaryl and Si(R₁₅)₃ groups, where the alkyl, cycloalkyl, alkenyl, cycloalkenyl, phenyl and heteroaryl groups are optionally substituted with one or more substituent(s) selected from the following group: alkyl, halogen, hydroxyl and alkoxy, where the alkyl and alkoxy is optionally substituted with halogen); where R₁₅ groups are selected, independently from each other, from the following scope: alkyl, cycloalkyl, alkenyl, cycloalkenyl, aryl and heteroaryl groups, where the alkyl, cycloalkyl, alkenyl, cycloalkenyl, aryl and heteroaryl groups are optionally substituted;
R₇, R₈ and R₉ are independently selected from the group consisting of H, D, F, Cl, Br, I, SF₅, alkyl, cycloalkyl, alkenyl, cycloalkenyl, aryl and heteroaryl groups, where the alkyl, cycloalkyl, alkenyl, cycloalkenyl and heteroaryl groups are optionally substituted,
with the proviso that
when R₁ to R₅, R′₁ to R′₅ and R₆ to R₉ are F, then R₁₀ is not pentafluorophenyl or methyl group;
when R₁ to R₅, R′₁ to R′₅ and R₆ to R₉ are H, then R₁₀ is not phenyl;
when R₁ to R₅, R′₁ to R′₅ are F and R₆ to R₉ are H, then Rio is not methyl;
when R₁ to R₅, R′₁, R′₃, R′₄, R′₅, R₆, R₇, R₈ are H, R′₂ and R₉ are Br, then R₁₀ is not Cl;
when R₁, R₅, R_{1′}, and R_{5′} are H, R₂, R₄, R_{2′} and R_{4′} are CF₃, R₆ and R₈ are F, and R₇ and R₉ are H, then R₁₀ is not Cl;
when R₁, R₅, R_{1′} and R_{5′} are H, R₂, R₄, R_{2′}, R_{4′}, R₆ and R₉ are CF₃, then R₁₀ is not H;
when R₁, R₂, R₄, R₅, R_{1′} R_{2′}, R_{4′} and R_{5′} are F, R_{3′}, R₃, R₆, R₇ and R₈ are H, R₉ is Cl, then R₁₀ is not 2-Br-phenyl;
when R₁, R₂, R₄, R₅, R_{1′} R_{2′}, R_{4′} and R_{5′} are F, R_{3′}, R₃, R₆, R₇, R₈ and R₉ are H, then R₁₀ is not CF₃.

2. Use of A catalyst for the partial reduction of a carbonyl group in an ester substrate or lactone substrate comprising a compound of general formula (I) according to claim 1
which substrate of said catalyst optionally contains one or more functional group(s) independently selected from the group consisting of non-carbonyl-conjugated olefinic bonds, non-carbonyl-conjugated acetylenic bonds, ether, amide, and halogen groups.

3. The compound according to claim 1, wherein the compound of formula (I) is characterized by general formula (Ia) 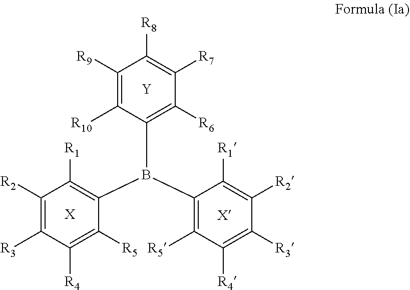
wherein
X ring and X′ ring are phenyl groups;
R₁ and R′₁ are independently selected from the group consisting of H, D and F;
R₅ and R′₅ are independently selected from the group consisting of H, D and F;
each R₂, R₃, R₄, R′₂, R′₃ and R′₄ are independently selected from the group consisting of H, D, F, Cl, Br, alkyl, cycloalkyl and aryl groups, where the alkyl, cycloalkyl and aryl groups are optionally substituted;
Y ring is phenyl group;
R₆ is selected from the group consisting of H, D and F;
R₁₀ is selected from the group consisting of Cl, Br, I, SF₅, alkyl, cycloalkyl and phenyl groups, where the alkyl, cycloalkyl and phenyl groups are optionally substituted with one or more substituent(s) selected from the following group: alkyl, halogen, hydroxyl and alkoxy, where the alkyl and alkoxy is optionally substituted with halogen);
R₇, R₈ and R₉ are independently selected from the group consisting of H, D, F, Cl, Br, alkyl and cycloalkyl groups, where the alkyl and cycloalkyl groups are optionally substituted.

4. The compound according to claim 3, wherein
X ring and X′ ring are phenyl groups, wherein each R₁, R′₁, R₅ and R′₅ are F; and each R₂, R₃ R₄, R′₂, R′₃ and R′₄ are independently selected from H and F;
Y ring is a phenyl group, wherein R₆ is selected from H and F; R₁₀ is selected from Cl, Br, methyl and pentafluorophenyl groups; and R₇, R₈ and R₉ are independently selected from H and F.

5. The compound according to claim 3, wherein X and X′ are independently selected from the group consisting of pentafluorophenyl, 2,3,4,6-tetrafluorophenyl, 2,3,5,6-tetrafluorophenyl, 2,4,6-trifluorophenyl, 2,3,6-trifluorophenyl, and 2,6-difluorophenyl groups.

6. The compound according to claim 3, wherein Y is selected from the group consisting of 2-chloro-6-fluorophenyl, 2-bromo-6-fluorophenyl, and perfluoro-1,1′-biphen-2-yl groups.

7. The compound according to claim 3, wherein the compound of general formula (I) is selected from the group consisting of the following compounds:
(2-bromo-6-fluorophenyl)bis(2,3,5,6-tetrafluorophenyl)borane (Compound 1);
(2-bromo-6-fluorophenyl)bis(2,3,6-trifluorophenyl)borane (Compound 2);
(2-bromo-6-fluorophenyl)bis(perfluorophenyl)borane (Compound 3);
(perfluoro-[1,1′-biphenyl]-2-yl)bis(2,4,6-trifluorophenyl)borane (Compound 4);
(2-bromo-6-fluorophenyl)bis(2,4,6-trifluorophenyl)borane (Compound 5);
(2-chloro-6-fluorophenyl)bis(2,3,5,6-tetrafluorophenyl)borane (Compound 6); and
(perfluoro-[1,1′-biphenyl]-2-yl)bis(2,3,5,6-tetrafluorophenyl)borane (Compound 7);
perfluoro-[1,1′-biphenyl]-2-yl)bis(2,3,6-trifluorophenyl)borane (Compound 8).

8. The compound according to claim 7, wherein the compound of general formula (I) is selected from the group consisting of the following compounds:
(2-bromo-6-fluorophenyl)bis(2,3,5,6-tetrafluorophenyl)borane (Compound 1);
(2-bromo-6-fluorophenyl)bis(2,3,6-trifluorophenyl)borane (Compound 2);
(2-bromo-6-fluorophenyl)bis(perfluorophenyl)borane (Compound 3);
(perfluoro-[1,1′-biphenyl]-2-yl)bis(2,4,6-trifluorophenyl)borane (Compound 4); and
(2-chloro-6-fluorophenyl)bis(2,3,5,6-tetrafluorophenyl)borane (Compound 6).

9. A method for the preparation of an aldehyde or a lactol by partial reduction of a carbonyl group in an ester substrate or lactone substrate, which substrate optionally contains one or more functional group(s) independently selected from the group consisting of non-carbonyl-conjugated olefinic bonds, non-carbonyl-conjugated acetylenic bonds, ether, amide, and halogen groups, wherein the method comprises the following steps:
a) said ester or lactone substrate is reacted with a silane in the presence of a catalytic amount of a compound of formula (I) defined in claim 1 to form a silyl acetal,
b) the thus-obtained silyl acetal is hydrolysed with one or more acidic or fluoride containing reagent(s) to form the aldehyde or lactol, and
c) optionally the obtained aldehyde or lactol is separated and purified.

10. A compound of general formula (II) 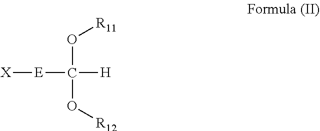
wherein
X is a halogen selected from the group consisting of Cl and Br;
E is either a (CH₂)ₘ or (CH₂)ₙ—O—(CH₂)ₚ, wherein m is an integer from 2 to 12, and n and p are, independently from each other, integers from 1 to 5, and any one of the methylene groups of (CH₂)ₘ or (CH₂)ₙ—O—(CH₂)ₚ may be optionally substituted with one or more substituent(s) independently selected from each other from the group consisting of halogens, optionally substituted alkyl groups (preferably methyl or trifluoromethyl groups) or optionally substituted alkoxy groups (preferably methoxy group);
R₁₁ is a trialkylsilyl or dialkylsiloxysilyl group, where the alkyl part is an optionally substituted C₁₋₆ alkyl group, preferably C₁₋₄ alkyl group;
R₁₂ is an optionally substituted alkyl group, preferably a C₁₋₆ alkyl group, preferably C₁₋₃ alkyl group.

11. The compound of formula (II) according to claim 10, wherein
X is a halogen selected from the group consisting of Cl and Br;
E is either a (CH₂)ₘ or (CH₂)ₙ—O—(CH₂)ₚ, wherein m is an integer from 2 to 10, and n and p are, independently from each other, integers from 1 to 3, and any one of the methylene groups of (CH₂)ₘ or (CH₂)ₙ—O—(CH₂)ₚ may be optionally substituted with 1 to 3 substituent(s), independently selected from each other from the group consisting of halogens, optionally substituted alkyl groups (preferably methyl group) or optionally substituted alkoxy groups (preferably methoxy group);
R₁₁ is a trialkylsilyl or dialkylsiloxysilyl group, where the alkyl part is a C₁₋₂ alkyl group, preferably triethylsilyl or dimethylsiloxysilyl group;
R₁₂ is a C₁₋₃ alkyl group, preferably methyl, ethyl, propyl or isopropyl group.

12. The compound of formula (II) according to claim 10, which is selected from the group consisting of the following compounds:
(4-bromo-1-ethoxybutoxy)triethylsilane (Example 14)
(3-bromo-1-ethoxypropoxy)triethylsilane (Example 15)
((5-bromo-1-ethoxypentyl)oxy)triethylsilane (Example 16)
((6-bromo-1-ethoxyhexyl)oxy)triethylsilane (Example 17)
(4-bromo-1-isopropoxybutoxy)triethylsilane (Example 18)
(2-(2-chloroethoxy)-1-ethoxyethoxy)triethylsilane (Example 19)
(2-(2-bromoethoxy)-1-ethoxyethoxy)triethylsilane (Example 21)
(4-bromo-1-ethoxy-2-fluorobutoxy)triethylsilane (Example 22)
((4-bromo-1-ethoxypentyl)oxy)triethylsilane (Example 24)
(4-bromo-1-ethoxy-2,2-difluorobutoxy)triethylsilane (Example 25)
(4-bromo-1-ethoxy-2-methylbutoxy)triethylsilane (Example 26).

13. A compound of general formula (III) 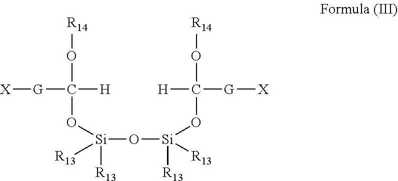
wherein
X is a halogen selected from the group consisting of Cl and Br;
G is either a (CH₂)ₘ or (CH₂)ₙ—O—(CH₂)ₚ, wherein m is an integer from 2 to 12, and n and p are, independently from each other, integers from 1 to 5, and any one of the methylene groups of (CH₂)ₘ or (CH₂)ₙ—O—(CH₂)ₚ may be optionally substituted with one or more substituent(s) [e.g. 1 to 5, or 1 to 4, or 1 to 3 or 1 or 2 substituent(s)], independently selected from each other from the group consisting of halogens, optionally substituted alkyl groups (preferably methyl or trifluoromethyl groups) or optionally substituted alkoxy groups (preferably methoxy group);
R₁₃ is an optionally substituted alkyl group, preferably a C₁₋₃₆ alkyl group, more preferably methyl group;
R₁₄ is an optionally substituted alkyl group, preferably a C₁₋₆ alkyl group, preferably C₁₋₃ alkyl group.

14. The compound of formula (III) according to claim 13, wherein
X is a halogen selected from the group consisting of Cl and Br;
G is either a (CH₂)ₘ or (CH₂)ₙ—O—(CH₂)ₚ, wherein m is an integer from 2 to 10, and n and p are, independently from each other, integers from 1 to 3, and any one of the methylene groups of (CH₂)ₘ or (CH₂)ₙ—O—(CH₂)ₚ may be optionally substituted with 1 to 3 substituent(s) [e.g. 1 or 2 substituent(s)], independently selected from each other from the group consisting of halogens, optionally substituted alkyl groups (preferably methyl group) or optionally substituted alkoxy groups (preferably methoxy group);
R₁₃ is a C₁₋₃ alkyl group, preferably methyl group;
R₁₄ is a C₁₋₃ alkyl group, preferably methyl, ethyl, propyl or isopropyl group.

15. The compound of formula (III) according to claim 13, which is 4,10-bis(3-bromopropyl)-6,6,8,8-tetramethyl-3,5,7,9,11-pentaoxa-6,8-disilatridecane (Example 27).
